SEQUENCE LISTING (26 sequences)

SEQ ID NO: 1 - DNA, 20 bp, Artificial Sequence
  Synthetic primer
atgccagcaagtccacagaa

SEQ ID NO: 2 - DNA, 20 bp, Artificial Sequence
  Synthetic primer
tgttctcgggcagacagttt

SEQ ID NO: 3 - DNA, 20 bp, Artificial Sequence
  Synthetic primer
caaagaggaacagcgggact

SEQ ID NO: 4 - DNA, 20 bp, Artificial Sequence
  Synthetic primer
acagccctcgcacatacttt

SEQ ID NO: 5 - DNA, 20 bp, Artificial Sequence
  Synthetic primer
gcagacagcacatggagaga

SEQ ID NO: 6 - DNA, 20 bp, Artificial Sequence
  Synthetic primer
gatccgattccgagcttccg

SEQ ID NO: 7 - DNA, 21 bp, Artificial Sequence
  Synthetic primer
tgcaccgccatatagaggaag

SEQ ID NO: 8 - DNA, 20 bp, Artificial Sequence
  Synthetic primer
tctgcagtgaactggcaatg

SEQ ID NO: 9 - DNA, 20 bp, Artificial Sequence
  Synthetic primer
accttgccagtttagctccc

SEQ ID NO: 10 - DNA, 20 bp, Artificial Sequence
  Synthetic primer
ttgggacctgcagtgaagaa

SEQ ID NO: 11 - DNA, 20 bp, Artificial Sequence
  Synthetic primer
gcagtcgcaacatgctcaag

SEQ ID NO: 12 - DNA, 20 bp, Artificial Sequence
  Synthetic primer
gggaacccttggggtcattt

SEQ ID NO: 13 - DNA, 20 bp, Artificial Sequence
  Synthetic primer
agaaacggaaacggcctcat

SEQ ID NO: 14 - DNA, 20 bp, Artificial Sequence
  Synthetic primer
catccaacgtggctctgagt

SEQ ID NO: 15 - DNA, 20 bp, Artificial Sequence
  Synthetic primer
atggagcaagtttggcagga

SEQ ID NO: 16 - DNA, 20 bp, Artificial Sequence
  Synthetic primer
gctgggaacagcggtagtat

SEQ ID NO: 17 - DNA, 20 bp, Artificial Sequence
  Synthetic primer
tccacagaacagctacccaa

SEQ ID NO: 18 - DNA, 20 bp, Artificial Sequence
  Synthetic primer
ccacagggctgcaattttcc

SEQ ID NO: 19 - DNA, 20 bp, Artificial Sequence
  Synthetic primer
ctgcgttaaagggggcacta

SEQ ID NO: 20 - DNA, 20 bp, Artificial Sequence
  Synthetic primer
acagccacggtgacaaagaa

SEQ ID NO: 21 - DNA, 20 bp, Artificial Sequence
  Synthetic primer
cttcatccttgctgggggtc

SEQ ID NO: 22 - DNA, 20 bp, Artificial Sequence
  Synthetic primer
ttgcggaggtcaacaccatt

SEQ ID NO: 23 - DNA, 23 bp, Artificial Sequence
  Synthetic primer
agaagctgtgctatgttgctcta

SEQ ID NO: 24 - DNA, 21 bp, Artificial Sequence
  Synthetic primer
tcaggcagctcatagctcttc

SEQ ID NO: 25 - DNA, 18 bp, Artificial Sequence
  Synthetic primer
ttcccgttcagctctggg

SEQ ID NO: 26 - DNA, 18 bp, Artificial Sequence
  Synthetic primer
ccctgcatccactggtgc

(19) United States
(12) Patent Application Publication
(10) Pub. No.: US 2019/0307749 A1
(43) Pub. Date: Oct. 10, 2019

(54) Mitochondria-Division Inhibitor 1 Protects Against Amyloid-B Induced Mitochondrial Fragmentation and Synaptic Damage in Alzheimer's Disease

(71) Applicant: Texas Tech University System, Lubbock, TX (US)

(72) Inventors: P. Hemachandra Reddy, Lubbock, TX (US); K. Chandra Sekhar, Kurnool (IN)

(21) Appl. No.: 16/380,211

(22) Filed: Apr. 10, 2019

Related U.S. Application Data
(60) Provisional application No. 62/655,677, filed on Apr. 10, 2018.

(51) Int. Cl. A61K 31/517; A61K 35/12; A61K 9/08; A61K 9/00

(52) U.S. Cl. CPC A61K 31/517; A61K 35/12; A61K 9/0029; A61K 9/0053; A61K 9/08

(57) ABSTRACT

The present invention includes a method for preventing or treating a disease or condition with excessive fragmentation of mitochondria or mitochondrial dysfunction comprising, consisting essentially of, or consisting of: identifying a subject suspected of needing treatment for excessive fragmentation of mitochondria or mitochondrial dysfunction; and administering to the subject with an amount of a mitochondrial division inhibitor 1 sufficient to prevent or treat the excessive fragmentation of mitochondria or mitochondrial dysfunction.

### CROSS-REFERENCE TO RELATED APPLICATIONS

This application claims priority to U.S. Provisional Application Ser. No. 62/655,677, filed Apr. 10, 2018, the entire contents of which are incorporated herein by reference.

### TECHNICAL FIELD OF THE INVENTION

The present invention relates in general to the field of mitochondria-division inhibitors, and more particularly, to novel water-soluble inhibitors with improved activity.

### STATEMENT OF FEDERALLY FUNDED RESEARCH

Not Applicable.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Apr. 10, 2019, is named TECH1180_SeqList.txt and is 6 bytes in size.

### BACKGROUND OF THE INVENTION

Without limiting the scope of the invention, its background is described in connection with diseases or conditions in which excessive fragmentation of mitochondria or mitochondrial dysfunction is found.

Alzheimer's disease (AD) is a progressive, age-dependent illness, characterized by the progressive decline of memory, cognitive function, and changes in behavior and personality [1-4]. Currently, over 46.8 million people worldwide, including 5.4 million Americans, live with AD-related dementia, and this number is estimated to increase to 131.5 million by 2050 [5]. AD-related dementia has huge economic consequences, with the total worldwide medical cost of dementia in 2015 estimated at $818 billion. By 2018, AD, including AD-related dementia, is expected to become a trillion-dollar disease [5]. With lifespan increasing in humans, AD is headed towards becoming the major health concern of elderly persons. Currently, there are no drugs or agents that can delay disease progression in elderly individuals and in patients with AD. Further, there are no definitive peripheral biomarkers that indicate disease in its early stages so that effective therapeutics can be initiated.

AD is largely associated with synaptic damage, mitochondrial structural and functional changes, inflammatory responses, hormonal imbalance, cell cycle changes, and neuronal loss [6-8]. In addition, there are 2 major pathological hallmarks: intracellular neurofibrillary tangles and extracellular amyloid-β (Aβ) deposits in the learning and memory regions of brain [3,9]. Genetic mutations in APP, PS1, and PS2 genes cause about 1-2% of the total number of AD cases. Several factors contribute to late-onset AD, including lifestyle, diet, environmental exposure, stroke, traumatic brain injury and multiple genetic variants in genetic loci, including sortilin-related receptor 1 gene clusterin, the complement component receptor 1, CD2AP, CD33, EPHA1, and MS4A4/MS4A6E genes and the ApoE4 genotype [7-8].

Despite many efforts toward treating AD, a need remains for compositions for use in preventing and/or treating the effects of AD at the molecular level.

### SUMMARY OF THE INVENTION

In one embodiment, the present invention includes a method for preventing or treating a disease or condition with excessive fragmentation of mitochondria or mitochondrial dysfunction comprising, consisting essentially of, or consisting of: identifying a subject suspected of needing treatment for excessive fragmentation of mitochondria or mitochondrial dysfunction; and administering to the subject with an amount of a mitochondrial division inhibitor 1 sufficient to prevent or treat the excessive fragmentation of mitochondria or mitochondrial dysfunction. In one aspect, the mitochondrial division inhibitor 1 comprises a phenolic OH group that enhances antioxidant/anti-inflammatory and water solubility. In another aspect, the mitochondrial division inhibitor 1 is modified to be at least partially soluble in water. In another aspect, the disease or condition with excessive fragmentation of mitochondria or mitochondrial dysfunction is Alzheimer's, Parkinson's, multiple sclerosis, amyotrophic lateral sclerosis, or Huntington's Disease. In another aspect, the method further comprises one or more pharmaceutically acceptable excipients, fillers, salts, or buffers. In another aspect, the disease is not associated with epilepsy and seizures, ischemia/reperfusion injury, oxygen glucose deprivation, or conditions associated with endosome aggregation and vesicle fusion during exocytosis. In another aspect, the mitochondrial division inhibitor 1 or active derivative thereof is administered to the subject at a dose of 1 to 120 mg/day/person. In another aspect, the mitochondrial division inhibitor 1 or active derivative thereof is administered to the subject at a dose of 10-60 mg daily. In another aspect, the mitochondrial division inhibitor 1 or active derivative thereof is administered to the subject via oral or parenteral administration. In another aspect, the mitochondrial division inhibitor 1 is 3-(2,4-Dichloro-5-methoxyphenyl)-2,3-dihydro-2-thioxo-4(1H)-quinazolinone, 3-(2,4-Dichloro-5-methoxyphenyl)-2-sulfanyl-4(3H)-quinazolinone, or 3-(2,4-dichloro-5-hydroxyphenyl)-2-thioxo-2,3-dihydroquinazolin-4(1H)-one. In another aspect, the mitochondrial division inhibitor 1 or active derivative thereof is a racemate, enantiomer, diastereomer, a mixture of enantiomer or a mixture of diastereomer. In another aspect, a pharmaceutically acceptable salt of mitochondrial division inhibitor 1 or active derivative thereof is formed from at least one of: an organic acid selected from formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phtalic acid, benzenesulphonic acid, toluenesulphonic acid, naphtalenesulphonic acid, or, methanesulphonic acid. In another aspect, the mitochondrial division inhibitor 1 or active derivative thereof is on the form of a tablets, capsules, pills, powders, granules, elixirs, tinctures, suspensions, syrups, emulsion, or formulated for intravenous administration.

In another embodiment, the present invention includes a method of treating or preventing memory loss in a subject suffering from a memory loss-related disease or aging, comprising, consisting essentially of, or consisting of, administering an effective amount of an amount of a mitochondrial division inhibitor 1 or active derivative thereof sufficient to prevent or treat the excessive fragmentation of mitochondria or mitochondrial dysfunction. In one aspect, the mitochondrial division inhibitor 1 or active derivative thereof is administered to the subject at a dose of 1 to 120 mg/day/person. In another aspect, the mitochondrial division inhibitor 1 or active derivative thereof is administered to the subject at a dose of 10-60 mg daily. In another aspect, the mitochondrial division inhibitor 1 or active derivative thereof is administered to the subject via oral or parenteral administration. In another aspect, the mitochondrial division inhibitor 1 is 3-(2,4-Dichloro-5-methoxyphenyl)-2,3-dihydro-2-thioxo-4(1H)-quinazolinone, 3-(2,4-Dichloro-5-methoxyphenyl)-2-sulfanyl-4(3H)-quinazolinone, or 3-(2,4-dichloro-5-hydroxyphenyl)-2-thioxo-2,3-dihydroquinazolin-4(1H)-one. In another aspect, the mitochondrial division inhibitor 1 or active derivative thereof is a racemate, enantiomer, diastereomer, a mixture of enantiomer or a mixture of diastereomer. In another aspect, a pharmaceutically acceptable salt of mitochondrial division inhibitor 1 or active derivative thereof is formed from at least one of: an organic acid selected from formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phtalic acid, benzenesulphonic acid, toluenesulphonic acid, naphtalenesulphonic acid, or, methanesulphonic acid. In another aspect, the memory loss-related disease is dementia. In another aspect, the dementia is Alzheimer's disease. In another aspect, a reduction in memory loss is in a cognitively normal older adult. In another aspect, the mitochondrial division inhibitor 1 or active derivative thereof is on the form of a tablets, capsules, pills, powders, granules, elixirs, tinctures, suspensions, syrups, emulsion, or formulated for intravenous administration.

In another embodiment, the present invention includes a method for protecting a subject from excessive fragmentation of mitochondria or mitochondrial dysfunction in neural cells comprising, consisting essentially of, or consisting of: identifying a subject suspected of needing treatment for excessive fragmentation of mitochondria or mitochondrial dysfunction in neurons, wherein the subject is suspected of having or being at risk for Alzheimer's Disease; and administering to the subject with an amount of a mitochondrial division inhibitor 1 sufficient to prevent the excessive fragmentation of mitochondria or mitochondrial dysfunction to prevent Alzheimer's Disease.

In another embodiment, the present invention includes a partially water-soluble mitochondrial division inhibitor 1 having the formula 3-(2,4-dichloro-5-hydroxyphenyl)-2-thioxo-2,3-dihydroquinazolin-4(1H)-one.

### BRIEF DESCRIPTION OF THE DRAWINGS

For a more complete understanding of the features and advantages of the present invention, reference is now made to the detailed description of the invention along with the accompanying figures and in which:

FIG. 1 is a flowchart that shows the experimental design of Mdivi1 and Aβ42 treatments in mouse neuroblastoma (N2a) cells.

FIGS. 2A to 2D show an analysis of mitochondrial dynamics, mitochondrial biogenesis and synaptic proteins. FIG. 2A shows a representative immunoblot of N2a cells treated with Aβ42, Mdivi1, Aβ42+Mdivi1, and Mdivi1+Aβ42 relative to untreated cells. FIG. 2B presents data from a quantitative densitometry analysis of mitochondrial dynamics proteins. FIG. 2C presents data from a quantitative densitometry analysis of mitochondrial biogenesis proteins. FIG. 2D presents data from a quantitative densitometry analysis of synaptic proteins.

FIG. 3 is a graph that shows Amyloid-β42 levels. Significantly reduced levels of Aβ42 levels were found in the N2a cells treated with Aβ42+Mdivi1 (P=0.01) and with Mdivi1+Aβ (P=0.04) relative to N2a cells treated only with Aβ42.

FIGS. 4A and 4B are graphs that show hydrogen peroxide levels. Hydrogen peroxide levels were analyzed in 2 ways: (FIG. 4A) the untreated N2a cells were compared with N2a cells treated Aβ42, Mdivi1, Aβ42+Mdivi1, and Mdivi1+Aβ42, and (FIG. 4B) Aβ42-treated N2a cells were compared to N2a cells treated with Aβ42+Mdivi1 and Mdivi1+Aβ42.

FIGS. 5A and 5B are graphs that show lipid peroxidation levels. Lipid peroxidation levels were analyzed in 2 ways: (FIG. 5A) the untreated N2a cells were compared with N2a cells treated Aβ42, Mdivi1, Aβ42+Mdivi1, and Mdivi1+Aβ42, and (FIG. 5B) Aβ42-treated N2a cells were compared to the N2a cells treated with Aβ42+Mdivi1 and Mdivi1+Aβ42.

FIGS. 6A and 6B are graphs that show Cytochrome c oxidase activity levels. Cytochrome c oxidase activity levels were analyzed in 2 ways: (FIG. 6A) the untreated N2a cells were with N2a cells treated Aβ42, Mdivi1, Aβ42+Mdivi1 and Mdivi1+Aβ42, and (FIG. 6B) the Aβ42-treated N2a cells were compared to the N2a cells treated with Aβ42+Mdivi1 and Mdivi1+Aβ42.

FIGS. 7A and 7B are graphs that show mitochondrial ATP levels. Mitochondrial ATP levels were analyzed in 2 ways: (FIG. 7A) the untreated N2a cells were compared with N2a cells treated with Aβ42, Mdivi1, Aβ42+Mdivi1, and Mdivi1+Aβ42, and (FIG. 7B) the Aβ42-treated N2a cells were compared to N2a cells treated with Aβ42+Mdivi1 and Mdivi1+Aβ42.

FIGS. 8A and 8B are graphs that show cell viability. Cell viability levels were analyzed in 2 ways: (FIG. 8A) the untreated N2a cells were compared with N2a cells treated Aβ42, Mdivi1, Aβ42+Mdivi1, and Mdivi1+Aβ42, and (FIG. 8B) the Aβ42-treated N2a cells were compared to N2a cells treated with Aβ42+Mdivi1 and Mdivi1+Aβ42.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present invention shows the protective and therapeutic effects of mitochondrial division inhibitor 1 (Mdivi1) in Alzheimer's disease (AD). Mdivi1 is hypothesized to reduce excessive fragmentation of mitochondria and mitochondrial dysfunction in AD neurons. Very little is known about whether Mdivi1 can confer protective effects in AD. In these studies, the inventors sought to determine the protective effects of Mdivi1 against amyloid-β (Aβ)- and mitochondrial fission protein, dynamin-related protein 1 (Drp1)-induced excessive fragmentation of mitochondria in AD progression. The inventors also studied preventive (Mdivi1+Aβ42) and intervention (Aβ42+Mdivi1) affects against Aβ42 in N2a cells. Using real-time RT-PCR and immunoblotting analysis, the inventors measured mRNA and protein levels of mitochondrial dynamics, mitochondrial biogenesis and synaptic genes. The inventors also assessed mitochondrial function by measuring H₂0₂, lipid peroxidation, cytochrome oxidase activity, and mitochondrial ATP. MTT assays were used to assess the cell viability. Aβ42 was found to impair mitochondrial dynamics, lower mitochondrial biogenesis, lower synaptic activity, and lower mitochondrial function. On the contrary, Mdivi1 enhanced mitochondrial fusion activity, lowered fission machinery, and increased biogenesis and synaptic proteins. Mitochondrial function and cell viability were elevated in Mdivi1-treated cells. Interestingly, Mdivi1 pre- and post-treated cells treated with Aβ showed reduced mitochondrial dysfunction, and maintained cell viability, mitochondrial dynamics, mitochondrial biogenesis, and synaptic activity. The protective effects of Mdivi1 were stronger in N2a+Aβ42 pre-treated with Mdivi1, than in N2a+Aβ42 cells than Mdivi1 post-treated cells, indicating that Mdivi1 works better in prevention than treatment in AD like neurons.

A dosage unit for use in a method of the present invention includes a mitochondrial division inhibitor 1 or active derivative thereof of the present invention, may be a single compound or mixtures thereof with other compounds, e.g., a potentiator. The compounds may be mixed together, form ionic or even covalent bonds. The mitochondrial division inhibitor 1 or active derivative thereof of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the mitochondrial division inhibitor 1 or active derivative thereof of the present invention to a patient in need of therapy. The mitochondrial division inhibitor 1 or active derivative thereof may also be administered as any one of known salt forms.

The mitochondrial division inhibitor 1 or active derivative thereof is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the mitochondrial division inhibitor 1 or active derivative thereof may be formulated to provide, e.g., maximum and/or consistent dosing for the particular form for oral, rectal, topical, intravenous injection or parenteral administration. While the mitochondrial division inhibitor 1 or active derivative thereof may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

For example, the mitochondrial division inhibitor 1 or active derivative thereof may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with a non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The mitochondrial division inhibitor 1 or active derivative thereof may be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like.

The mitochondrial division inhibitor 1 or active derivative thereof may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the mitochondrial division inhibitor 1 or active derivative thereof may be coupled one or more biodegradable polymers to achieve controlled release of the mitochondrial division inhibitor 1 or active derivative thereof, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the mitochondrial division inhibitor 1 or active derivative thereof and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagous, trachea, lungs and alveoli, the mitochondrial division inhibitor 1 or active derivative thereof may also be delivered as an intranasal form via use of a suitable intranasal vehicle. For dermal and transdermal delivery, the mitochondrial division inhibitor 1 or active derivative thereof may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of mitochondrial division inhibitor 1 or active derivative thereof may include the following forms.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of cancer, which comprise one or more containers containing a pharmaceutical composition comprising a therapeutically effective amount of mitochondrial division inhibitor 1 or active derivative thereof. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

As used herein, the term “chewable” refers to semi-soft, palatable and stable chewable treat without addition of water. It should be appreciated to the skilled artisan that a chewable composition will be stable and palatable, fast disintegrating, semi-soft medicated chewable tablets (treats) by extrusion without the addition of extraneous water. A soft chewable tablets does not harden on storage and are resistant to microbial contamination. A semi-soft chewable contain a blend of any one or more of binders, flavours, palatability enhancers, humectants, disintegrating agents, non-aqueous solvents, and diluents that are plasticized with liquid plasticizers, such as glycols and polyols to make them ductile and extrudable. The chewable can be made by extrusion, e.g., including fats or lipids as plasticizers and binding agents, is manufactured in the absence of added water, uses plasticizers to replace water in extrudable matrices, contains humectants to maintain the extrudable chew in a pliant and soft state during its shelf life, or any combination thereof. The chewable form may be provided in conjunction with one or more flavorants and/or taste masking agents that improve the taste of the formulation greater than 10, 20, 30, 40, 50, 60, 70, 80, or 90%. The chewable can include the active agent and the ion exchange resin to enhance taste masking.

For topical administration, the composition can be incorporated into creams, ointments, gels, transdermal patches and the like. The composition can also be incorporated into medical dressings, for example wound dressings e.g. woven (e.g. fabric) dressings or non-woven dressings (e.g. gels or dressings with a gel component). The use of alginate polymers in dressings is known, and such dressings, or indeed any dressings, may further incorporate the alginate oligomers of the invention.

Generally, the formula of the mitochondrial division inhibitor 1 is:

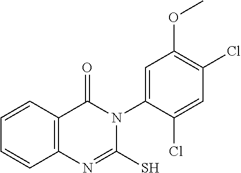 [SMILES: COc1cc(-n2c(S)nc3ccccc3c2=O)c(Cl)cc1Cl]

As taught herein, the present invention also includes an improvement to the solubility of the mitochondrial division inhibitor 1 (Mdivi1), which is an active derivative in which the modified Mdivi1 structure has a phenolic OH group that enhances antioxidant/anti-inflammatory properties and increases solubility. The at least partially water-soluble inhibitor has the formula: 3-(2,4-dichloro-5-hydroxyphenyl)-2-thioxo-2,3-dihydroquinazolin-4(1H)-one. One advantage of the partially water-soluble Mdivi1 is that the use of the toxic compound dimethyl sulfoxide (DMSO) can be avoided. Because of safety concerns, clinical trials with DMSO have been stopped. DMSO is known to have toxicity of the eye, induces headaches, causes a burning and itching on contact with the skin, and triggers strong allergic reactions.

The present inventors recognized that mitochondrial abnormalities are largely involved in AD progression. For example, AD is associated with: (1) increased free radical production, lipid peroxidation, oxidative DNA damage, oxidative protein damage, and decreased ATP production in postmortem AD brains, compared to brains from age-matched healthy subjects [10-14]; in AD transgenic mice; and in cell lines that express mutant APP or cells treated with Aβ [15-16], (2) studies of mitochondrial enzyme activities found decreased levels of cytochrome oxidase activity, pyruvate dehydrogenase, and α-ketodehydrogenase in fibroblasts, lymphoblasts, and postmortem brains from AD patients, compared to neurons, fibroblasts, and lymphoblasts from age-matched healthy subjects [16], (3) mitochondrial DNA changes were higher in postmortem brain tissue from AD patients and aged-matched healthy subjects, compared to DNA changes in postmortem brain tissue from young, healthy subjects, suggesting that the accumulation of mitochondrial DNA in AD pathogenesis is age-related [17-18], (4) several groups investigated mitochondrial gene expressions in postmortem AD brains and in brain specimens from AD transgenic mice [19-21]. It was found that mitochondrially encoded genes abnormally expressed in the brains AD patients and AD mice. A recent, time-course global gene expression study in an AD mouse model (Tg2576) and age-matched non-transgenic littermates revealed an up-regulation of mitochondrial genes in the Tg2576 mice, suggesting that mitochondrial metabolism is impaired by mutant APP/Aβ and that the up-regulation of mitochondrial genes may be a compensatory response to mutant APP/Aβ [20]; further, Manczak and colleagues found an abnormal expression of mitochondrially encoded genes in postmortem AD brains compared to the brains of healthy subjects, suggesting impaired mitochondrial metabolism in AD [22], 5) recent mitochondrial studies in brain tissue from AD patients and neuronal cells expressing mutant APP found that Aβ fragments mitochondria and causes structural changes in neurons from AD patients [21,23-27].

Recent Aβ and mitochondrial studies found impaired mitochondrial dynamics (excessive mitochondrial fragmentation and reduced fusion) in AD postmortem brains and AD cell and mouse models [21,23-25,26-31]. Studies by the inventors revealed that Aβ interacts with Drp1, with a subsequent increase in free radical production, which in turn activates Drp1 and Fis1, and causes excessive mitochondrial fragmentation, defective transport of mitochondria to synapses, low synaptic ATP, and synaptic dysfunction in AD neurons [21,24]. Given the increase in free radical production and excessive fragmentation in mitochondria that are involved in AD, the inventors hypothesize that drugs capable of reducing free radicals or decreasing excessive mitochondrial fragmentation may be effective therapeutic approaches to treat AD.

Excessive mitochondrial fragmentation is well documented in neurodegenerative diseases, including Alzheimer's, Parkinson's, and Huntington's, leading to impaired mitochondrial dynamics, defective axonal transport, low synaptic ATP and, ultimately, synaptic damage [32,33]. Based on excessive mitochondrial fragmentation, impaired mitochondrial dynamics, defective axonal transport, low synaptic ATP, and synaptic damage that are present in AD, it has been proposed that mitochondrial division inhibitors are potential candidates to treat excessive mitochondrial fragmentation and its associated factors in AD progression and pathogenesis.

To identify mitochondrial fission inhibitors, several groups have independently screened chemical libraries and have found several inhibitors: Mdivi1 [34], Dynasore [35], and P110 [36]. Among these, Mdivi1 has been extensively investigated with experimental rodent models of epilepsy and seizures [37], ischemia/reperfusion injury [38-41], oxygen glucose deprivation [42], and such conditions as aggregation of endosomes and vesicle fusion during exocytosis [43]. In all of these diseased states and conditions, Mdivi1 was found to have beneficial effects on affected tissues and cells, by reducing excessive mitochondrial fission and maintaining the fission-fusion balance in mitochondria and the normal functioning of cells.

Mdivi1 is a cell-permeable, selective mitochondrial fission inhibitor; its molecular weight is 353.22. It inhibits GTPase Drp1 activity by blocking the self-assembly of Drp1, resulting in reversible formation of elongated and tubular mitochondria in wild-type cells [34]. However, it is unclear whether Mdivi1 reduces Aβ-induced excessive mitochondrial fragmentation, maintains and/or enhances mitochondrial function and synaptic activities in Aβ-treated N2a cells.

In the present study, the inventors determined the protective effects of Mdivi1 against Aβ-induced excessive mitochondrial fragmentation and synaptic toxicities in mouse neuroblastoma (N2a) cells. Using Aβ42 peptide and Mdivi1 and mouse neuroblastoma (N2a) cells, the present inventors measured (1) mRNA and protein levels of mitochondrial fission and fusion genes, biogenesis genes, and synaptic genes; (2) mitochondrial function by measuring H₂O₂, lipid peroxidation, cytochrome oxidase activity, and mitochondrial ATP; and (3) cell viability.

Chemicals and Reagents: The Aβ42 peptide was purchased from Anaspec (Fremont, Calif., USA); Water soluble Mdivi1 was made in our laboratory and N2a cells were purchased from American Type Culture Collection (ATCC) (Manassas, Va., USA). Dulbecco's Modified Eagle Medium (DMEM) and Minimum Essential Medium (MEM), penicillin/streptomycin, Trypsin-EDTA, and fetal bovine serum were purchased from GIBCO (Gaithersberg, Md., USA).

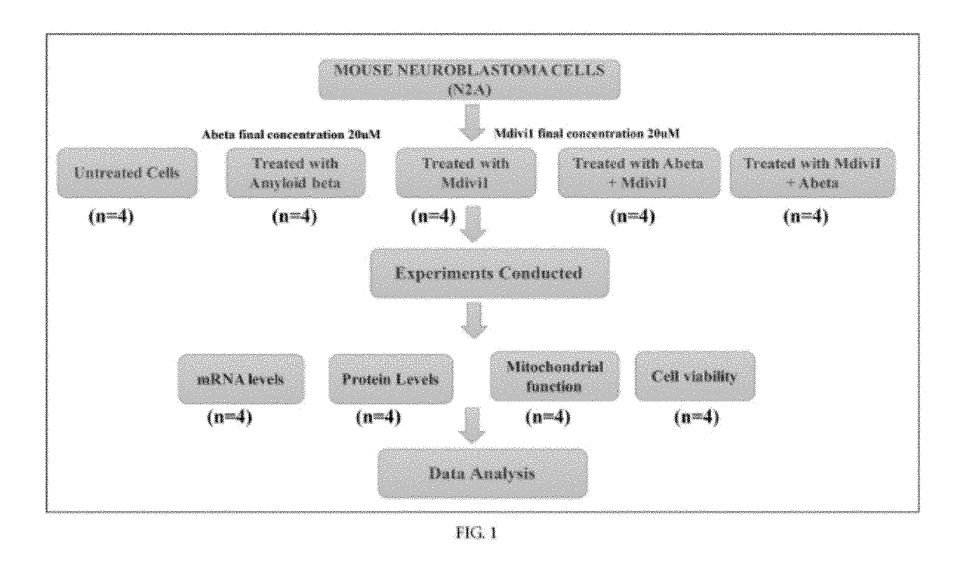

Tissue culture: The N2a cells were grown for 3 days in a serum-free medium (1:1 mixture of DMEM and OptiMEM, plus penicillin and streptomycin [Invitrogen, Carlsbad, Calif., USA]) until the cells developed neuronal processes. As shown in FIG. 1, these cells were used for 5 groups—one control group and 4 treatment groups: (1) untreated N2a cells (the control group), (2) N2a cells treated (incubated) with the Aβ42 peptide (Aβ42 treatment group; 20 μM final concentration) for 6 hrs, (3) N2a cells treated with Mdivi1 for 24 hrs (Mdivi1 treatment group, 20 M final concentration), (4) N2a cells treated with the Aβ42 peptide for 6 hrs then treated with Mdivi1 for 24 hrs (Aβ42+Mdivi1 treatment group), and (5) N2a cells treated with Mdivi1 for 24 hrs and then treated with the Aβ42 peptide for 6 hrs (Mdivi1+Aβ42 treatment group). As shown in FIG. 1, the inventors performed 4 independent cell cultures and treatments for all experiments (n=4).

The N2a cells from all 5 groups (4 treatment groups and 1 control group) were harvested, and their cell pellets were collected and used for quantitative real-time RT-PCR, immunoblotting analysis of mitochondrial and synaptic proteins, and mitochondrial functional assays for hydrogen peroxide production, lipid peroxidation, cytochrome c oxidase activity, and MTT determination. Isolated mitochondria from N2a cells from all 5 groups were used to determine mitochondrial ATP as described in Reddy et al. [6]. As shown in FIG. 1, the inventors performed 4 independent cell cultures and treatments for each parameter—levels of mRNA, proteins, mitochondrial functional assays for lipid peroxidation, cytochrome c oxidase activity, ATP production, and cell viability.

Quantitative real-time RT-PCR: Using the reagent TriZol (Invitrogen), total RNA was isolated from cell pellets from the 5 N2a cell groups (FIG. 1). Using primer express software (Applied Biosystems, Carlsbad, Calif., USA), the inventors designed the oligonucleotide primers for the housekeeping genes 3-actin, GAPDH, mitochondrial structural genes, fission genes (Drp1, Fis1), fusion genes (MFN1, MFN2, Opa1), biogenesis genes (PGC1c, Nrf1, Nrf2, TFAM), and synaptic genes (synaptophysin and PSD95) (see Table 1 for primer sequences and amplicon sizes). Using SYBR-Green chemistry-based quantitative real-time RT-PCR, mRNA expressions of the above-mentioned genes were measured, as described by Manczak and Reddy [44].

TABLE 1
Summary of quantitative real-time RT-PCR oligonucleotide primers used in
measuring mRNA expression in mitochondrial dynamics, mitochondrial biogenesis,
and synaptic genes in in N2a cells treated with Aβ42, Mdivi1 + Aβ42,
Aβ42 + Mdivi1 relative to untreated N2a cells

| | | | |
|---|---|---|---|
| | | PCR | |
| Gene | DNA Sequence (5′-3′) | Product Size | SEQ ID NO: |

Mitochondrial Dynamics Genes

| | | | |
|---|---|---|---|
| Drp1 | Forward Primer ATGCCAGCAAGTCCACAGAA | 86 | 1 |
| | Reverse Primer TGTTCTCGGGCAGACAGTTT | | 2 |
| Fis1 | Forward Primer CAAAGAGGAACAGCGGGACT | 95 | 3 |
| | Reverse Primer ACAGCCCTCGCACATACTTT | | 4 |
| MFN1 | Forward Primer GCAGACAGCACATGGAGAGA | 83 | 5 |
| | Reverse Primer GATCCGATTCCGAGCTTCCG | | 6 |
| MFN2 | Forward Primer TGCACCGCCATATAGAGGAAG | 78 | 7 |
| | Reverse Primer TCTGCAGTGAACTGGCAATG | | 8 |
| Opa1 | Forward Primer ACCTTGCCAGTTTAGCTCCC | 82 | 9 |
| | Reverse Primer TTGGGACCTGCAGTGAAGAA | | 10 |

Mitochondrial Biogenesis Genes

| | | | |
|---|---|---|---|
| PGC1α | Forward Primer GCAGTCGCAACATGCTCAAG | 83 | 11 |
| | Reverse Primer GGGAACCCTTGGGGTCATTT | | 12 |
| Nrf1 | Forward Primer AGAAACGGAAACGGCCTCAT | 96 | 13 |
| | Reverse Primer CATCCAACGTGGCTCTGAGT | | 14 |
| Nrf2 | Forward Primer ATGGAGCAAGTTTGGCAGGA | 96 | 15 |
| | Reverse Primer GCTGGGAACAGCGGTAGTAT | | 16 |
| TFAM | Forward Primer TCCACAGAACAGCTACCCAA | 84 | 17 |
| | Reverse Primer CCACAGGGCTGCAATTTTCC | | 18 |

Synaptic Genes

| | | | |
|---|---|---|---|
| Synaptophysin | Forward Primer CTGCGTTAAAGGGGGCACTA | 81 | 19 |
| | Reverse Primer ACAGCCACGGTGACAAAGAA | | 20 |
| PSD95 | Forward Primer CTTCATCCTTGCTGGGGGTC | 90 | 21 |
| | Reverse Primer TTGCGGAGGTCAACACCATT | | 22 |

Housekeeping Genes

| | | | |
|---|---|---|---|
| Beta Actin | Forward Primer AGAAGCTGTGCTATGTTGCTCTA | 91 | 23 |
| | Reverse Primer TCAGGCAGCTCATAGCTCTTC | | 24 |
| GAPDH | Forward Primer TTCCCGTTCAGCTCTGGG | 59 | 25 |
| | Reverse Primer CCCTGCATCCACTGGTGC | | 26 |

The mRNA transcript level was normalized against β-actin and the GAPDH at each dilution. The standard curve was the normalized mRNA transcript level, plotted against the log-value of the input cDNA concentration at each dilution. To compare β-actin, GAPDH, and relative quantification was performed according to the (CT) method (Applied Biosystems). Briefly, this method involved averaging triplicate samples, which were taken as the CT values for β-actin, GAPDH, and neuroprotective markers. β-actin normalization was used in the present study because the β-actin CT values were similar for the control and the 4 treatment groups in terms of mitochondrial dynamics, biogenesis, and synaptic genes. The ΔCT-value was obtained by subtracting the average β-actin CT value from the average CT-value of the synaptic mitochondrial ETC genes and the mitochondrial structural genes. The ΔCT of untreated cells was used as the calibrator. The fold change was calculated according to the formula 2\hat{ }-(Δ ΔCT), where ΔΔCT is the difference between ΔCT and the ΔCT calibrator value. To determine the statistical significance of mRNA expression, the CT value differences between the untreated cells and other experimental groups of cells were used in relation to β-actin normalization. Statistical significance was calculated, using one-way ANOVA. Fold changes of mRNA were compared 2 ways—comparison 1, untreated N2a cells with 1) N2a+Mdivi1, 2) N2a+Aβ42, 3) N2a+Aβ42+Mdivi1, 4) N2a+Mdivi1+Aβ42, and comparison 2, N2a+Aβ42 with 1) N2a+Aβ42+Mdivi1 and 2) N2a+Mdivi1+Aβ42.

Immunoblotting analysis: To determine the effects of Mdivi1 and Aβ42 at the protein levels of mitochondrial dynamics and biogenesis and synaptic genes that exhibited altered mRNA expression, the inventors performed immunoblotting analyses of protein lysates isolated from cell pellets of all 5 groups of cells (FIG. 1), as described in Reddy et al. [6]. Twenty g of protein was resolved from each group of cells on a 4-12% Nu-PAGE gel (Invitrogen). The resolved proteins were transferred to nylon membranes (Novax Inc., San Diego, Calif., USA) and were then incubated for 1 hour at room temperature with a blocking buffer (5% dry milk dissolved in a TBST buffer). The PVDF membranes were incubated overnight with primary antibodies (see Table 2) and washed 3 times with a TBST buffer at 10-minute intervals. They were then incubated for 2 hours with appropriate secondary antibodies, followed by 3 additional washes at 10-minute intervals. Mitochondrial and synaptic proteins were detected with cheminilumniscence reagents (Pierce Biotechnology, Rockford, Ill., USA), and the bands from immunoblots were quantified on a Kodak scanner, following manufacturer's instructions (ID Image Analysis Software, Kodak Digital Science, Kennesaw, Ga., USA), and the gel images were analyzed from gel images captured with a Kodak digital science CD camera. The lanes were marked to define the positions and specific regions of the bands. An ID fine-band command was used to locate and to scan the bands in each lane and to record the readings.

TABLE 2
Summary of antibody dilutions and conditions used in the immunoblotting analysis of
mitochondrial dynamics, mitochondrial biogenesis, and synaptic proteins in N2a cells
treated with Aβ42, Mdivi1 + Aβ42, Aβ42 + Mdivi1 relative to untreated N2a cells

| | Primary Antibody - | Purchased from Company, | Secondary Antibody, | Purchased from Company, |
|---|---|---|---|---|
| Marker | Species and Dilution | City & State | Dilution | City & State |
| Drp1 | Rabbit Polyclonal | Novus Biological, | Donkey Anti-rabbit | GE Healthcare Amersham, |
| | 1:500 | Littleton, CO | HRP 1:10,000 | Piscataway, NJ |
| Fis1 | Rabbit Polyclonal | MBL International | Donkey Anti-rabbit | GE Healthcare Amersham, |
| | 1:500 | Corporation-life. | HRP 1:10,000 | Piscataway, NJ |
| | | Woburn, MA | | |
| Mfn1 | Rabbit Polyclonal | Novus Biological, | Donkey Anti-rabbit | GE Healthcare Amersham, |
| | 1:400 | Littleton, CO | HRP 1:10,000 | Piscataway, NJ - |
| Mfn2 | Rabbit Polyclonal | Abcam, | Donkey Anti-rabbit | GE Healthcare Amersham, |
| | 1:400 | Cambridge, MA | HRP 1:10,000 | Piscataway, NJ |
| OPA1 | Rabbit Polyclonal | Novus Biological, | Donkey anti-rabbit | GE Healthcare Amersham, |
| | 1:500 | Littleton, CO | HRP 1:10,000 | Piscataway, NJ |
| SYN | Rabbit Monoclonal | Abcam, | Donkey Anti-rabbit | GE Healthcare Amersham, |
| | 1:400 | Cambridge, MA | HRP 1:10,000 | Piscataway, NJ |
| PSD95 | Rabbit Monoclonal | Abcam, | Donkey Anti-rabbit | GE Healthcare Amersham, |
| | 1:300 | Cambridge, MA | HRP 1:10,000 | Piscataway, NJ |
| PGC1α | Rabbit Polyclonal | Novus Biological, | Donkey Anti-rabbit | GE Healthcare Amersham, |
| | 1:500 | Littleton, CO | HRP 1:10,000 | Piscataway, NJ |
| Nrf1 | Rabbit Polyclonal | Santa Cruz Biotechnology, | Donkey Anti-rabbit | GE Healthcare Amersham, |
| | 1:300 | Inc. | HRP 1:10,000 | Piscataway, NJ |
| | | Dallas, TX | | |
| Nrf2 | Rabbit Polyclonal | Novus Biological, | Donkey Anti-rabbit | GE Healthcare Amersham, |
| | 1:300 | Littleton, CO | HRP 1:10,000 | Piscataway, NJ |
| TFAM | Rabbit Polyclonal | Novus Biological, | Donkey Anti-rabbit | GE Healthcare Amersham, |
| | 1:300 | Littleton, CO | HRP 1:10,000 | Piscataway, NJ |
| B-actin | Mouse Monoclonal | Sigma-Aldrich, | Sheep Anti-mouse | GE Healthcare Amersham, |
| | 1:500 | St Luis, MO | HRP 1:10,000 | Piscataway, NJ |

Measurement of soluble Aβ42 levels in untreated N2a cells and N2a cells treated with Mdivi1. The cell pellets were washed several times with PBS buffer and washed pellets were used and measured soluble Aβ42 levels in the N2a cells+Aβ42, Aβ42+Mdivi1, and Mdivi1+Aβ42 treatment groups following method described in Manczak et al [23]. Briefly, protein lysates from untreated control N2a cells and lysates from 4 treatment groups were homogenized in a Tris-buffered saline (pH 8.0) containing protease inhibitors (20 mg/ml pepstatin A, aprotinin, phophsoramidon, and leupeptin; 0.5 mM phenylmethanesulfonyl fluoride and 1 mM ethyleneglycol-bis(flaminoethyl ether)-NN tetraacetic acid). Samples were sonicated briefly and centrifuged at 10,000 g for 20 min at 4° C. The soluble fraction was used to determine the soluble Aβ by ELISA. For each sample, Aβ1-40 and Aβ1-42 were measured with commercial colorimetric ELISA kits (Biosource International, Camarillo, Calif., USA) that were specific for each species. A 96-well plate reader was used, following the manufacturer's instructions. Each sample was run in duplicate. Protein concentrations of the homogenates were determined following the BSA method, and Aβ was expressed as pg Aβ/mg protein.

Mitochondrial functional assays. H₂O₂ production: Using an Amplex® Red H₂O₂ assay kit (Molecular Probes, Eugene, Oreg., USA), H₂O₂ production was measured using cell pellets, as described in Manczak and Reddy [44]. Briefly, H₂O₂ production was measured in the protein lysates prepared from cell pellets of control untreated N2a cells and 4 treatment groups (FIG. 1). A BCA protein assay kit (Pierce Biotechnology) was used to estimate protein concentration. The reaction mixture contained mitochondrial proteins (μg/μl), Amplex Red reagents (50 μM), horseradish peroxidase (0.1 U/ml), and a reaction buffer (1×). The mixture was incubated at room temperature for 30 minutes, followed by spectrophotometer readings of fluorescence (570 nm). Finally, H₂O₂ production was determined, using a standard curve equation expressed in nmol/μg mitochondrial protein. H₂O₂ levels were compared 2 ways—comparison 1, untreated N2a cells with 1) N2a+Mdivi1, 2) N2a+Aβ42, 3) N2a+Aβ42+Mdivi1, 4) N2a+Mdivi1+Aβ42, and comparison 2, N2a+Aβ42 with 1) N2a+Aβ42+Mdivi1 and 2) N2a+Mdivi1+Aβ42.

Lipid peroxidation assay: Lipid peroxidates are unstable indicators of oxidative stress in the brain. The final product of lipid peroxidation is 4-hydroxy-2-nonenol (HNE), which was measured in the N2a cell lysates prepared from cell pellets of control and treatment groups. The inventors used an HNE-His ELISA kit (Cell BioLabs, Inc., San Diego, Calif., USA), as described in Manczak and Reddy [44]. Briefly, freshly prepared protein as added to a 96-well protein binding plate and incubated overnight at 4° C. The protein then washed 3 times with a buffer. After the last wash, the anti-HNE-His antibody was added to the protein in the wells, incubated for 2 hours at room temperature, and then washed again 3 times. The samples were then incubated with a secondary antibody conjugated with peroxidase for 2 hours at room temperature, followed by incubation with an enzyme substrate. Optical density was measured (at 450 nm) to quantify the level of HNE. Lipid peroxidation levels were compared 2 ways—comparison 1, untreated N2a cells with 1) N2a+Mdivi1, 2) N2a+Aβ42, 3) N2a+Aβ42+Mdivi1, 4) N2a+Mdivi1+Aβ42, and comparison 2, N2a+Aβ with 1) N2a+Aβ42+Mdivi1 and 2) N2a+Mdivi1+Aβ42.

Cytochrome c oxidase activity: Cytochrome oxidase activity was measured in each of the 5 groups of N2a cells. Enzyme activity was assayed spectrophotometrically using a Sigma Kit (Sigma-Aldrich) following manufacturer's instructions. Briefly, 2 μg protein lysate was added to 1.1 ml of a reaction solution containing 50 μl 0.22 mM ferricytochrome c that was fully reduced by sodium hydrosulphide, Tris-HCl (pH 7.0), and 120 mM potassium chloride. The decrease in absorbance at 550 mM was recorded for 1-min reactions at 10-sec intervals. Cytochrome oxidase activity was measured according to the following formula: mU/mg total mitochondrial protein=(A/min sample−(A/min blank)×1.1 mg protein×21.84). The protein concentrations were determined following the BCA method. Cytochrome oxidase activity levels were compared 2 ways—comparison 1, untreated N2a cells with 1) N2a+Mdivi1, 2) N2a+Aβ42, 3) N2a+Aβ42+Mdivi1, 4) N2a+Mdivi1+Aβ42, and comparison 2, N2a+Aβ42 with 1) N2a+Aβ42+Mdivi1 and 2) N2a+Mdivi1+Aβ42.

ATP levels: ATP levels were measured in N2a cell mitochondria from the treatment groups using an ATP determination kit (Molecular Probes). A bioluminescence assay was used, based on the reaction of ATP with recombinant firefly luciferase and its substract luciferin. Luciferase catalyzes the formation of light from ATP and luciferin. It is the emitted light that is linearly related to the concentration of ATP, which is measured with a luminometer. ATP levels were measured from mitochondrial pellets using a standard curve method. ATP levels were compared 2 ways—comparison 1, untreated N2a cells with 1) N2a+Mdivi1, 2) N2a+Aβ42, 3) N2a+Aβ42+Mdivi1, 4) N2a+Mdivi1+Aβ42, and comparison 2, N2a+Aβ42 with 1) N2a+Aβ42+Mdivi1 and 2) N2a+Mdivi1+Aβ42.

Statistical considerations. Statistical analyses were conducted for mitochondrial structural and functional parameters in the N2a cells from the 5 experimental groups, using one-way ANOVA with Dunnett correction. The parameters included H₂O₂, cytochrome oxidase activity, lipid peroxidation, ATP production, and cell viability. To determine the effect of Mdivi1 on N2a cells, in the absence and presence of Aβ42, the inventors analyzed and compared data in 2 ways—comparison 1, untreated N2a cells with 1) N2a+Mdivi1, 2) N2a+Aβ42, 3) N2a+Aβ42+Mdivi1, 4) N2a+Mdivi1+Aβ42, and comparison 2, N2a+Aβ with 1) N2a+Aβ+Mdivi1 (curative) and 2) N2a+Mdivi1+Aβ42 (preventive).

mRNA expressions of mitochondrial dynamics genes. Amyloid-β42 treatment: In the N2a cells treated with Aβ42 compared to untreated N2a cells, mRNA expression levels were significantly higher: in the fission Drp1 by 1.4 fold (P=0.02) and Fis1 by 1.4 fold (P=0.03) (Table 3). In contrast, mRNA expression levels of mitochondrial fusion genes were lower but not significant—Mfn1 by −1.2 fold, Mfn2 by −1.3 fold, and Opa1 by −1.2 fold. These findings indicate the presence of abnormal mitochondrial dynamics in cells treated with Aβ.

Mdivi1: The mRNA levels of N2a cells treated with Mdiv1 were significantly lower in the fission genes Drp1 (1.5-fold decrease, P=0.01 and Fis1 (1.3-fold decrease) and higher for the fusion genes Mfn1 by 1.3 fold, Mfn2 by 1.2 fold, and Opa1 by 1.2 fold (Table 3).

Treatment with Aβ42 and Mdivi1: In the N2a cells treated with Aβ42 and then treated with Mdivi1, the mRNA levels were unchanged for Drp1 and Fis1 and for Mfn1, Mfn2 and Opa1 and CypD, compared to the mRNA levels of untreated N2a cells (Table 3). The mRNA levels of N2a cells treated with Mdivi1 and then treated with Aβ42 did were significantly higher for the fusion genes Mfn1 by 2.1 fold (P=0.01), Mfn2 by 1.7 fold (P=0.03), and Opa1 by 1.9 fold (P=0.01) (Table 3).

Mitochondrial biogenesis genes. Aβ42: To determine the effects of Aβ42 and Mdivi1 on mitochondrial biogenesis genes, mRNA expression levels of PGC1a, Nrf1, Nrf2, and TFAM genes were measured. Significantly lower mRNA expressions were found in the biogenesis genes from N2a cells treated with Aβ42 relative to the mRNA expression level of untreated cells:—PGC1α by 5.8 fold (P=0.001), Nrf1 by 2.0 fold (P=0.01), Nrf2 by 2.1 fold (P=0.01), and TFAM by 2.5 fold (P=0.01) (Table 3).

Mdivi1: mRNA levels were significantly increased for PGC1α by a 2.2-fold (P=0.01), Nrf1 by a 2.2 fold (P=0.01), Nrf2 by 1.6 fold (P=0.03), and TFAM by a 1.5 fold (P=0.03) in Mdivi-treated cells relative to untreated cells (Table 3). These observations indicate that Mdivi1 increases mitochondrial biogenesis activity.

Aβ42+Mdivi1: In cells treated with Aβ42 first and then treated with Mdivi1, mRNA expression levels were unchanged for Nrf1, Nrf2 and only slightly higher for PGC1α (by 1.4 fold) and TFAM (1.2 fold) (Table 3).

Mdivi1+Aβ42: In cells treated with Mdivi1 and then treated with Aβ42, levels of mRNA expression were slightly higher for biogenesis genes: PGC1α by 1.1 fold, Nrf1 by 1.3 fold, Nrf2 by 1.3, and TFAM by 1.3 (Table 3). These results suggest that Mdivi1 treatment prevented Aβ42-induced biogenesis toxicity.

Synaptic genes. Aβ42: In cells treated with Aβ42 compared to untreated cells, mRNA expression levels were lower for synaptophysin by 1.4 fold (P=0.04) and PSD95 by 2.6 fold (P=0.004), indicating that Aβ42 reduces synaptic activity (Table 3).

Mdivi1: mRNA levels were significantly higher for PSD95 5.1 fold (P=0.004) and higher for synaptophysin by 1.3 fold in the Mdivi1-treated cells (Table 3). These findings indicate that Mdivi1 boosts synaptic activity in healthy cells.

Aβ42+Mdivi1: In cells treated with Aβ42 and then treated with Mdivi1, mRNA levels were significantly higher for PSD95 by 4.8 fold (P=0.001) and slightly higher for synaptophysin 1.2 fold (Table 3). These observations suggest that Mdivi1 rescued synaptic activity from Aβ42-induced toxicity.

Mdivi1+Aβ: In cells treated with Mdivi1 and then treated with Aβ42, significantly higher mRNA expression levels were found for synaptophysin by 1.7 fold (P=0.01) and PSD95 by 1.5 fold (P=0.04) (Table 3), indicating that Mdivi1 prevented Aβ42-induced synaptic activity.

Comparison to Aβ42-treated N2a cells: As shown in Table 3, mRNA expression levels of fission genes were lower in N2a cells treated with Aβ42+Mdivi1 (Drp1 by 1.5 fold, P=0.03; Fis1 by 1.7 fold, P=0.02), and with Mdivi1+Aβ42 (Drp1 by 1.5 fold, P=0.02; Fis1 by 1.6 fold, P=0.03) relative to the expression levels of fission genes in N2a cells treated only with Aβ42. In contrast, fusion genes were higher in the N2a cells treated with Aβ42+Mdivi1 (Mfn1 by 1.6 fold, P=0.04; Mfn2 by 1.6 fold, P=0.03; Opa1 by 1.3) and Mdivi1+Aβ42 (Mfn1 by 2.6 fold, P=0.003; Mfn2 by 2.2 fold, P=0.01; Opa1 by 2.3 fold, P=0.002) than the N2a cells treated only with Aβ42.

TABLE 3
mRNA fold changes in N2a cells treated with Aβ42 and Mdivi1

| | mRNA fold changes compare with untreated cells | | | | mRNA fold changes compare with Aβ42 treated cells | |
|---|---|---|---|---|---|---|
| Genes | Mdivi1 | Aβ42 | Aβ42 + Mdivi1 | Mdivi1 + Aβ42 | Aβ42 + Mdivi1 | Mdivi1 + Aβ42 |

Mitochondrial Structural genes

| | | | | | | |
|---|---|---|---|---|---|---|
| Drp1 | −1.5* | 1.4* | −1.2 | −1.1 | −1.5* | −1.5* |
| Fis1 | −1.3 | 1.4* | −1.2 | −1.2 | −1.7* | −1.6* |
| Mfn1 | 1.3 | −1.2 | 1.3 | 2.1* | 1.6* | 2.6** |
| Mfn2 | 1.2 | −1.3 | 1.2 | 1.7* | 1.6* | 2.2* |
| OPA1 | 1.2 | −1.2 | 1.0 | 1.9* | 1.3 | 2.3* |

Mitochondrial Biogenesis Genes

| | | | | | | |
|---|---|---|---|---|---|---|
| PGC1α | 2.2* | −5.8** | 1.4 | 1.1 | 8.1*** | 6.5** |
| Nrf1 | 2.2* | −2.0* | 1.0 | 1.3 | 2.0* | 2.7** |
| Nrf2 | 1.6* | −2.1* | 1.0 | 1.3 | 2.0* | 2.7** |
| TFAM | 1.5* | −2.5* | 1.2 | 1.3 | 2.9** | 3.2** |

Synaptic Genes

| | | | | | | |
|---|---|---|---|---|---|---|
| Synaptophysin | 1.3 | −1.4* | 1.2 | 1.7* | 1.5* | 2.2* |
| PSD95 | 5.1** | −2.6* | 4.8** | 1.5* | 8.6*** | 3.8** |

*P ≤ 0.05
**P ≤ 0.005
***P ≤ 0.0005

Mitochondrial biogenesis genes were greater in the N2a cells treated with Aβ42+Mdivi1 (PGC1α by 8.1 fold, P=0.0001; Nrf1 by 2.0 fold, P=0.01, Nrf2 by 2.0 fold, P=0.01, TFAM by 2.9 fold, P=0.004) and Mdivi1+Aβ42 (PGC1α by 6.5 fold, P=0.001; Nrf1 by 2.7 fold, P=0.003; Nrf2 by 2.7 fold, P=0.004; TFAM by 3.2 fold, P=0.002) than in the N2a cells treated only with Aβ42, indicating that Mdivi1 increases Aβ42-induced reduced biogenesis activity. Similarly, synaptic genes were greater in N2a cells treated with Aβ42+Mdivi1 (synaptophysin by 1.5 fold, P=0.04 and PSD95 by 8.6 fold, P=0.0002) and Mdivi1+Aβ42 (synaptophysin 2.2 fold, P=0.01 and PSD95 by 3.8 fold, P=0.003) than in N2a cells treated only with Aβ42 (Table 3).

Immunoblotting analysis. To determine the effects of Aβ42 and Mdiv1 on mitochondrial proteins, the inventors quantified mitochondrial proteins in 3 independent treatments of N2a cells with Aβ42, Mdivi1, Aβ42+Mdivi1, and Mdivi1+Aβ42.

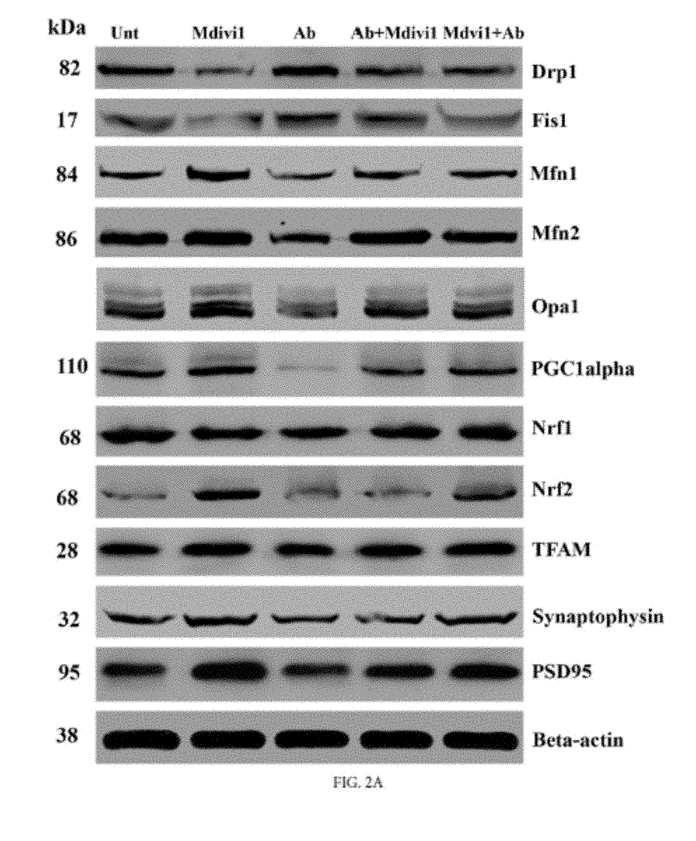

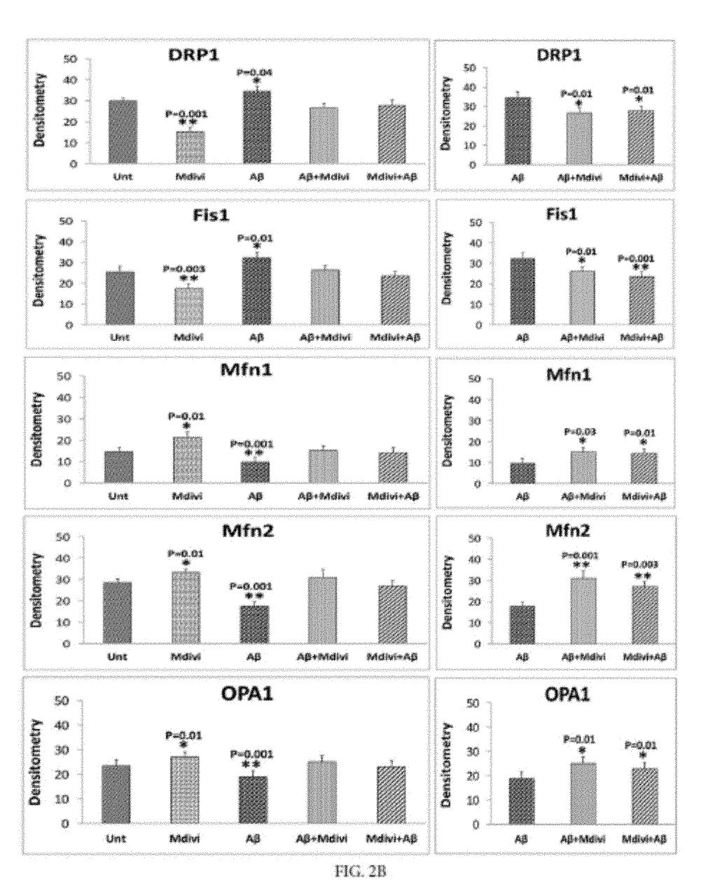

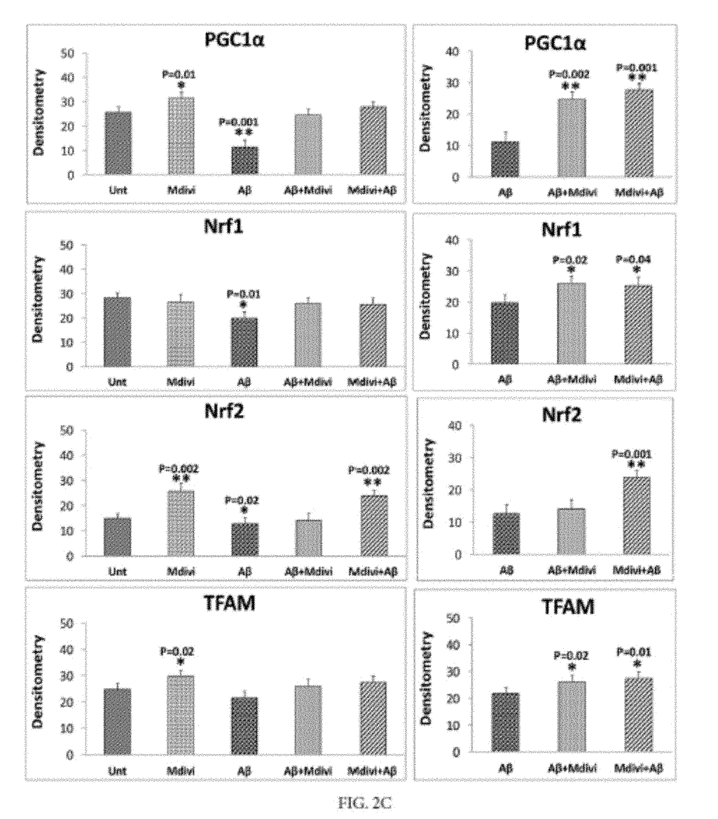

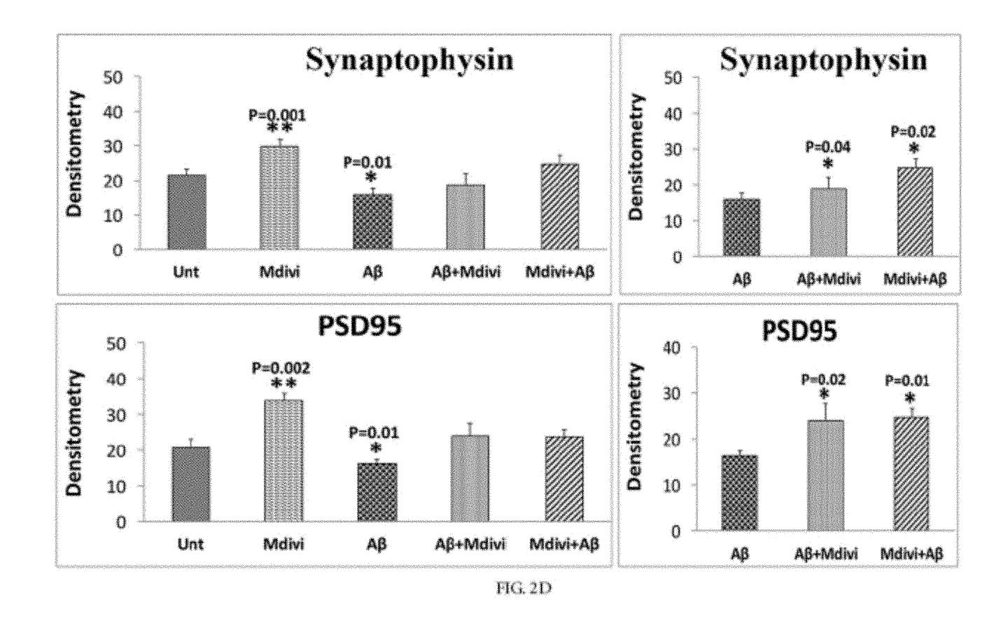

Comparison to untreated N2a cells: In N2a cells treated with Aβ42, significantly higher increased proteins levels were found for Drp1 (P=0.04) and Fis1 (P=0.01) (FIGS. 2A and 2B) compared to untreated N2a cells. In contrast, significantly lower levels of Mfn1 (P=0.001), Mfn2 (P=0.001), and Opa1 (P=0.001) were found in N2a cells treated with Aβ42. Significantly lower levels of PGC1α (P=0.001), Nrf1 (P=0.01) and Nrf2 (P=0.02) were also found in the Aβ42-treated N2a cells (FIGS. 2A and 2C), similar to synaptophysin (P=0.01) and PSD95 (P=0.01), which were significantly lower in the Aβ42 treated N2a cells, compared to untreated N2a cells (FIGS. 2A and 2D).

Drp1 (P=0.001) and Fis1 (P=0.003) protein levels were significantly lower, in contrast to Mfn1 (P=0.01), Mfn2 (P=0.01) and OPA1 (P=0.01) protein levels were significantly higher in N2a cells treated with Mdivi1 (FIGS. 2A and 2B). Interestingly, PGC1α (P=0.01), Nrf2 (P=0.002), and TFAM (P=0.02) were also significantly higher in Mdivi1-treated cells (FIGS. 2A and 2C), suggesting increased biogenesis activity. Levels of synaptophysin (P=0.001) and PSD95 (P=0.002) were significantly higher in Aβ-treated cells than were they in untreated N2a cells (FIGS. 2A and 2D).

Unchanged protein level for Drp1 and Fis1 were in N2a cells treated with Aβ42+Mdivi1, compared to levels of these proteins in untreated N2a cells (FIGS. 2A and 2B). Also unchanged protein levels for Drp1 and Fis1 were found in the N2a cells treated with Mdivi1+Aβ42 treated cells, compared to levels of these proteins in untreated N2a cells (FIGS. 2A and 2B). Overall, these findings suggest that Mdivi1 reduces fission activity in the N2a cells, and Mdivi1 enhances fusion and biogenesis activities in the presence of Aβ42.

Comparison to Aβ42-treated cells: As shown in FIGS. 2A and 2B, significantly lower levels of Drp1 (P=0.01) and Fis1 (P=0.01) were found in N2a cells treated with Aβ42+Mdivi1 relative to Aβ42 treated N2a cells, and significantly lower levels of proteins (Drp1, P=0.01; Fis1, P=0.001) were found in N2a cells treated with Mdivi1+Aβ42 relative to the Aβ42-treated N2a cells. In contrast, significantly higher levels of Mfn1 (P=0.03), Mfn2 (P=0.001), and Opa1 (0.01) were found in N2a cells treated with Aβ42+Mdivi1 relative to Aβ42 treated N2a cells and significantly higher levels of Mfn1 (P=0.01), Mfn2 (P=0.003) and Opa1 (P=0.01) were also found in Mdivi1+Aβ42-treated N2a cells relative to Aβ42 treated N2a cells.

Also significantly higher levels of PGC1α (P=0.002), Nrf1 (P=0.02) and TFAM (P=0.02) were found in N2a cells treated with Aβ42+Mdivi1 relative to Aβ42 treated N2a cells, similar to PGC1α (P=0.001), Nrf1 (P=0.04), Nrf2 (P=0.001), and TFAM (P=0.01) N2a cells that were treated with Mdivi1+Aβ42 (FIGS. 2A and 2C), indicating that Mdivi1 enhances biogenesis in the presence of Aβ42. Likewise, levels of synaptophysin and PSD95 were significantly higher in the N2a cells treated with Aβ42+Mdivi1 (P=0.04 and P=0.02, respectively) as were they when treated with Mdivi1+Aβ42 (P=0.02 and P=0.01 respectively), compared to the Aβ42 treated cells (FIGS. 2A and 2D). These results indicate that Mdivi1 treatment enhances synaptic activity in the presence of Aβ42.

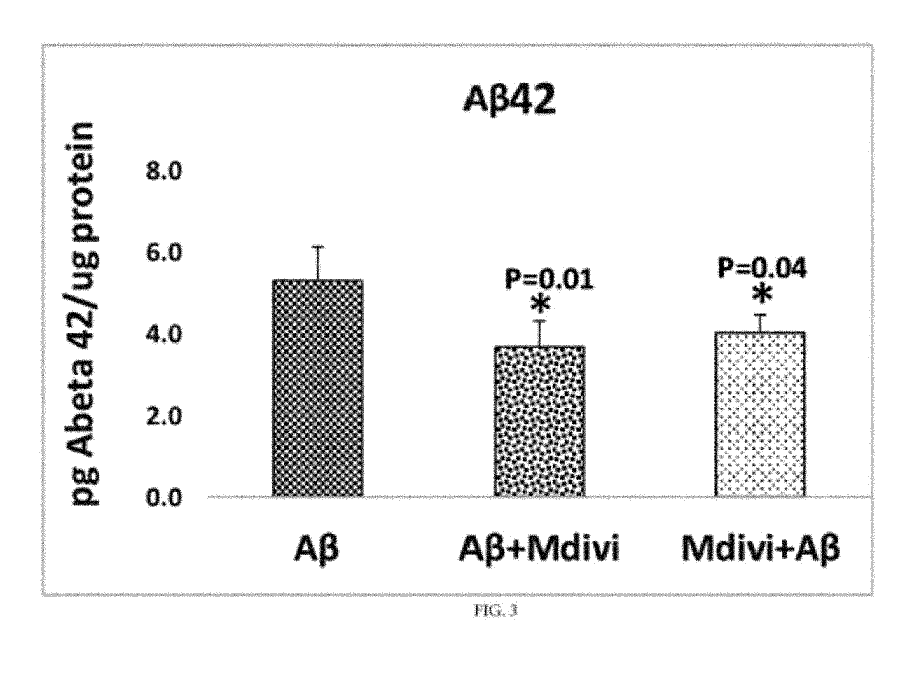

Mdivi1 and levels of Aβ42. To determine whether Mdivi1 lowers Aβ42 levels in N2a cells, using Sandwich ELISA the inventors measured Aβ42 levels in N2a cells treated with Aβ42+Mdivi1 (curative) and Mdivi1+Aβ42 (preventive). As shown in FIG. 3, significantly lower levels of Aβ42 levels were found in the N2a cells treated with Aβ42+Mdivi1 (P=0.01) and with Mdivi1+Aβ (P=0.04), compared to N2a cells treated only with Aβ42.

These observations indicate that Mdivi1 reduces Aβ42 levels via mitochondrial dynamics pathway since Aβ42 was found to be lower in Mdivi1+Aβ42 and Aβ42+Mdivi1 proteins, regardless of the sequence of treatment, as long as Mdivi1 was involved in the treatment. Also, since Aβ42 were found to be higher in N2a cells, regardless of the sequence of treatment, as long as Mdivi1 was involved in the treatment, mitochondrial fragmentation lowered, as did the level of free radicals.

Mitochondrial function. Parameters of mitochondrial function were studied in Aβ-treated N2a cells (n=4) to determine whether affects mitochondrial function and whether Mdivi1 confers preventive effects on Mdivi1 in the presence or absence of the Aβ42 peptide. The parameters included H₂O₂ production, cytochrome oxidase activity, lipid peroxidation, ATP production, and cell viability. The inventors compared the data 2 ways—in comparison 1—untreated N2a cells with Mdivi1, Aβ42, Mdivi1+Aβ42 and Mdivi1+Aβ42 (preventive) and in comparison 2—Aβ42 with Aβ42+Mdivi1 and Mdivi1+Aβ42 (curative).

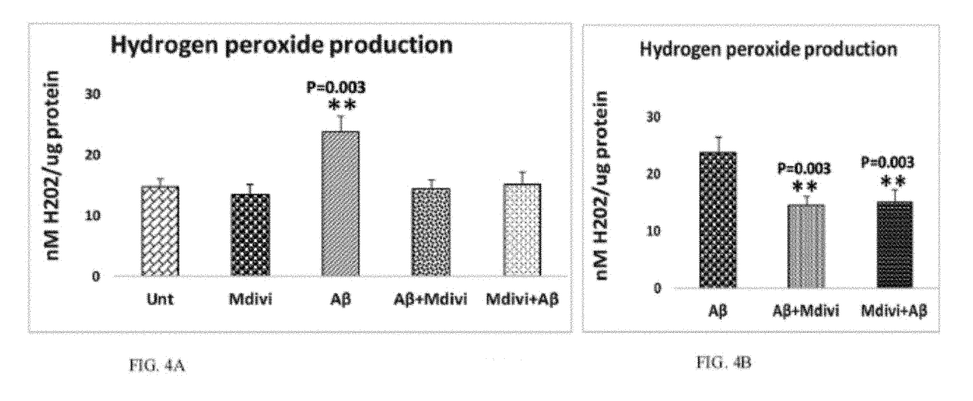

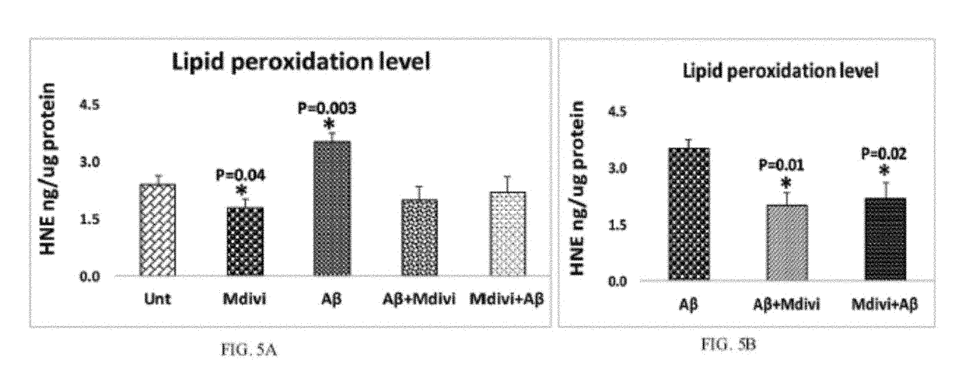

H₂O₂ production: In comparison 1—as shown in FIG. 4A, significantly increased levels of H₂O₂ were found in mitochondria from N2a cells treated with Aβ42 (P=0.003), but H₂O₂ levels were unchanged in mitochondria isolated from cells treated with Aβ42+Mdivi1 and Mdivi1+Aβ42. H₂O₂ levels significantly lower in Aβ+Mdivi1 (P=0.003) and Mdivi1+Aβ (P=0.003) than N2a+Aβ42 cells. These results suggest that Mdivi1 reduces H₂O₂ levels in the presence of Aβ42 in N2a cells (FIG. 4B). Lipid peroxidation: To determine whether Aβ affects lipid peroxidation in N2a cells that underwent Aβ42 incubation, 4-hydroxy-2-nonenol, an indicator of lipid peroxidation, was measured. Significantly higher levels of lipid peroxidation (P=0.003) were found in the treated (FIG. 5A, 5B) relative to the untreated N2a cells. However, significantly lower levels of lipid peroxidation (P=0.04) were found in the Mdivi1-treated N2a cells relative to cells that were not treated with Aβ42. Significantly lower levels of lipid peroxidation were found in N2a cells treated with Aβ42+Mdivi1 (P=0.01) and Mdivi1+Aβ42 (P=0.02) relative to N2a cells treated only with Aβ42. These findings suggest that Aβ42 increases lipid peroxidation, on the contrary Mdivi1 reduces lipid peroxidation in N2a cells. Further, Mdivi1 reduces lipid peroxidation in the presence of Aβ42.

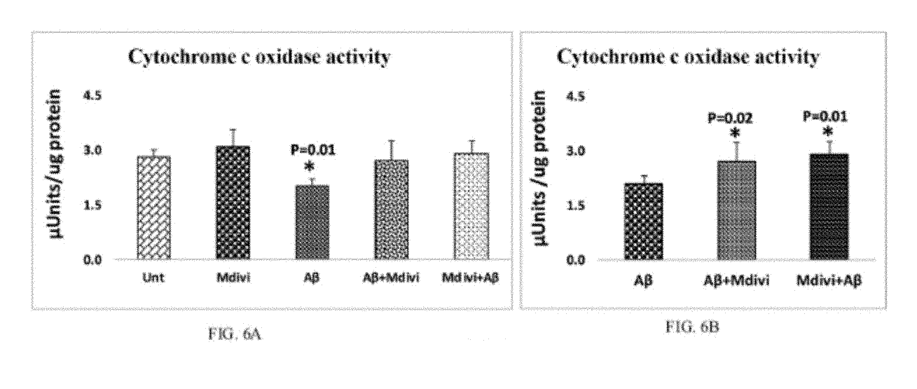

Cytochrome c oxidase activity: Significantly lower levels of cytochrome oxidase activity were found in N2a cells treated with Aβ42 (P=0.01) (FIG. 6A). However, increased levels of were found in the Mdivi1, Aβ42+Mdivi1, and Mdivi1+Aβ treated N2a cells, but levels were not significant. As shown in FIG. 6B, significantly increased cytochrome oxidase activity was found in the N2a cells treated with Aβ42+Mdivi1 (P=0.02) and Mdivi1+Aβ (P=0.01) compared cells treated with Aβ42 alone, indicating that Mdivi1 prevent toxic effects of Aβ42 on cytochrome oxidase activity.

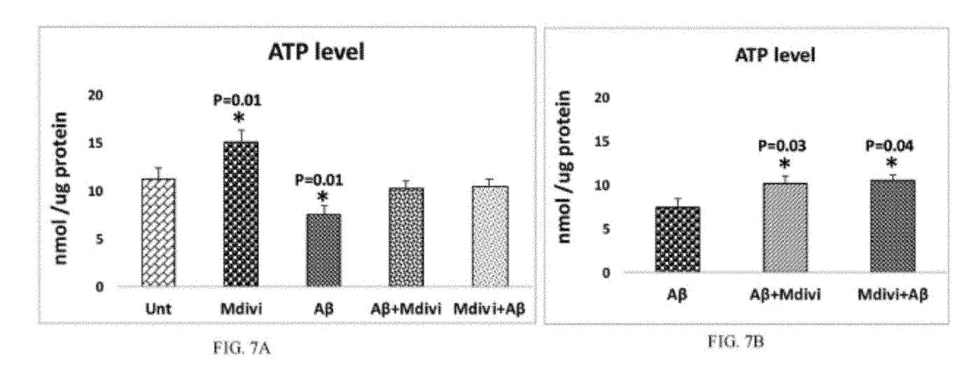

ATP production: Significantly lower levels of ATP were found in N2a cells that were treated with Aβ42 (P=0.01) (FIG. 7A). Significantly increased levels of ATP were found in N2a cells treated with Mdivi1 alone (P=0.01) (FIG. 7A). Significantly higher ATP levels were found in the N2a cells treated with Aβ42+Mdivi1 (P=0.03) and Mdivi1+Aβ42 (P=0.04), indicating that Mdivi1 enhances ATP levels in the presence of Aβ42 (FIG. 7B).

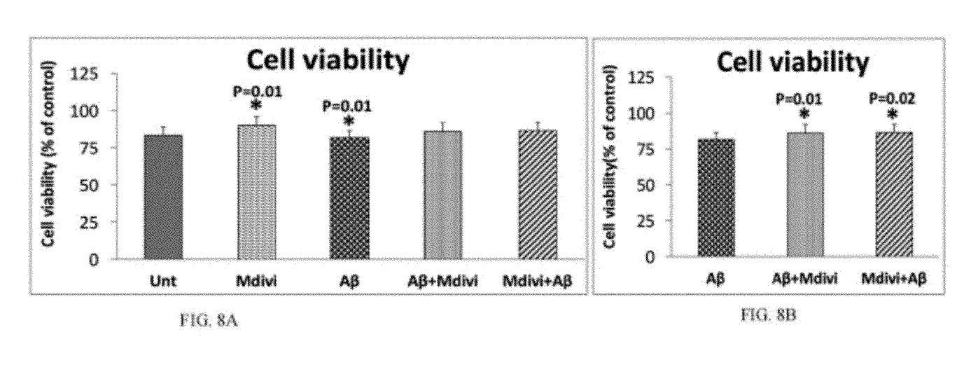

Cell viability: Significantly lower levels of cell viability were found in N2a cells treated with Aβ42 (0.01) (FIG. 8A), but cell viability was significantly higher in N2a cells treated with Mdivi1 (P=0.01). As shown in FIG. 8 B, cell viability was also higher in the N2a cells treated with Aβ+Mdivi1 (P=0.01) and Mdivi1+Aβ42 (P=0.02) relative to N2a+Aβ42, indicating that Mdivi1 prevents a decrease in cell viability that Aβ causes.

N2a cells treated with Aβ42 were found to impair mitochondrial dynamics (increased fission and decreased fusion), reduced biogenesis, and decreased synaptic activity and mitochondrial function. On the other hand, Mdivi1 reduced fission machinery and enhanced fusion activity and also increased biogenesis and synaptic proteins. Mitochondrial function and cell viability were elevated in Mdivi1 treated cells. Interestingly, N2a cells treated with Mdivi1 and Aβ42 (Mdiv1+Aβ42 and Aβ42+Mdiv1) showed reduced mitochondrial dysfunction and maintained cell viability, mitochondrial dynamics, mitochondrial biogenesis, and synaptic activity

The protective effects of water soluble Mdivi1 were stronger in pre-treated cells than post-treated cells—in other words, Mdivi1 works better in prevention than treatment in AD like neurons, however, it is still able to treat AD. In the current study, impaired mitochondrial dynamics was evident in the Aβ42-treated N2a cells (Table 3). These findings agree with earlier studies [23,35-26,45] in which increased mitochondrial fission and reduced fusion were reported. Mitochondrial biogenesis genes were reduced in Aβ42-treated cells, indicating that Aβ42 reduces biogenesis activity. Synaptic activity was significantly reduced in Aβ42 treated N2a cells. Protein data confirmed mRNA changes, suggesting that Aβ42 affects both mRNA and proteins of mitochondria and synapses in AD like neurons. On the other hand, in cells treated with Mdivi1—mitochondrial biogenesis and synaptic genes were increased and mitochondrial fission genes were reduced and fusion genes were increased, indicating that Mdivi1 treatment is beneficial to cells. Further, when the inventors compared mRNA and protein data of N2a cells+Aβ42 with ‘Aβ42+Mdivi1 (curative)’ and ‘Mdivi1+Aβ42 (prevention)’, protective effects of preventive were stronger than Mdivi1—In other words, in the presence of Aβ42 in N2a cells, treatment of Mdivi1 before Aβ42 treatment did stronger than after Aβ42 followed by Mdivi1 treatment (see Table 3). These observations strongly suggest that Mdivi1 prevents mitochondrial structural, biogenesis and synaptic genes from expressing abnormally. Overall, Mdivi1 protects mitochondrial structure and mitochondrial function by regulating mitochondrial fission and fusion genes in AD.

Aβ42 levels were lower in N2a cells treated with Mdivi1. Interestingly, Mdivi1 reduces Aβ42 in both pre- and post-Mdivi1 treated cells in the presence of Aβ42. Based on current study findings, it appears that Mdivi1 may reduce excessive mitochondrial fragmentation and increase mitochondrial fusion activity, leading to reduced production of mitochondria-generated excessive free radicals, increased cell viability, and maintained mitochondrial/neuronal function. The toxicity caused by Aβ42 [16,20.46] is expected to be reduced by Mdivi1 treatment in N2a cells. However, further research is still needed to understand how Mdivi1 reduces Aβ42 levels in N2a cells.

In the current study, the inventors found mitochondrial function and cell viability were reduced in Aβ42 treated cells. These observations concur with previous studies on Aβ-induced defective mitochondrial function and cell viability [6,23,26,45-49]. On the other hand, Mdivi1-treated cells exhibited enhanced mitochondrial function—increased mitochondrial ATP, cytochrome oxidase activity and cell viability, and reduced free radicals and oxidative stress. These observations strongly suggest that Mdivi1 reduces cellular toxicity and boosts mitochondrial function.

Next, the inventors determined the protective effects of Mdivi1, particularly ‘reduced fragmentation of mitochondria’ and increased and/or maintained mitochondrial function and cell viability in the presence of Aβ42 in cells. The inventors measured mitochondrial functional parameters and cell viability in N2a cells treated with Mdivi1 in the presence or absence of Aβ42. As described in the results section, Aβ42-induced excessive mitochondrial fragmentation and defective mitochondrial function and cell viability were reversed in Mdivi1-treated cells. The reversal effect was stronger in Mdivi1 pre-treated cells than Mdivi1 post-treated cells (see Table 3), indicating that Mdivi1 acts as strong preventive drug for AD. These findings are consistent with mitochondrial and synaptic gene-expression and protein data. In the presence of Aβ42, Mdivi1 reduced free radicals and lipid peroxidation, and increased mitochondrial ATP and cytochrome oxidase activity and cell viability. Thus, these data clearly show that Mdivi1 protects cells from Aβ42 toxicity.

In summary, Mdivi1 treatment confers protective effects against Aβ42-induced mitochondrial and synaptic toxicities in N2a cells. Findings from our study may have implications for other neurological diseases, such as Huntington's [50-53], Parkinson's [54-56], multiple sclerosis [57], and ALS [58], in which excessive mitochondrial fragmentation is present.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

[1] Reddy P H, Manczak M, Mao P, Calkins M J, Reddy A P and Shirendeb U (2010) Amyloid-beta and mitochondria in aging and Alzheimer's disease: implications for synaptic damage and cognitive decline. J Alzheimers Dis 20, S499-512.
[2] Mattson M P (2004) Pathways towards and away from Alzheimer's disease. Nature 430, 631-639.
[3] LaFerla F M, Green K N, Oddo S (2007) Intracellular amyloid-beta in Alzheimer's disease. Nat Rev Neurosci 8, 499-509.
[4] Swerdlow R H (2007) Pathogenesis of Alzheimer's disease. Clin Interv Aging 2, 347-59.
[5] World Alzheimer's Report, 2015 (https://www.alz.co.uk/research/WorldAlzheimerReport2015.pdf)
[6] Reddy P H, Manczak M, Yin X, Grady M C, Mitchell A, Kandimalla R, Kuruva C S (2016) Protective effects of a natural product, curcumin, against amyloid 3 induced mitochondrial and synaptic toxicities in Alzheimer's disease. J Investig Med 64, 1220-1234.
[7] Reddy P H, Tripathi R, Troung Q, Tirumala K, Reddy T P, Anekonda V, Shirendeb U P, Calkins M J, Reddy Aβ, Mao, P., et al. (2012) Abnormal mitochondrial dynamics and synaptic degeneration as early events in Alzheimer's disease: implications to mitochondria-targeted antioxidant therapeutics. Biochim Biophys Acta 1822, 639-649.
[8] Mao P, Reddy P H (2011) Aging and amyloid beta-induced oxidative DNA damage and mitochondrial dysfunction in Alzheimer's disease: implications for early intervention and therapeutics. Biochim Biophys Acta 1812, 1359-1370.
[9] Selkoe D J (2001) Alzheimer's disease: genes, proteins, and therapy. Physiol Rev 81, 741-766.
[10] Gibson G E, Sheu K F, Blass J P (1998) Abnormalities of mitochondrial enzymes in Alzheimer disease. J Neural Transm (Vienna) 105, 855-870.
[11] Parker W D Jr, Filley C M, Parks J K (1990) Cytochrome oxidase deficiency in Alzheimer's disease. Neurology 40, 1302-1303.
[12] Maurer I, Zierz S Möller H J (2000) A selective defect of cytochrome c oxidase is present in brain of Alzheimer disease patients. Neurobiol Aging 21, 455-462.
[13] Smith M A, Perry G, Richey P L, Sayre L M, Anderson V E, Beal M F, Kowall N (1996) Oxidative damage in Alzheimer's. Nature 382, 120-121.
[14] Devi L, Prabhu B M, Galati D F, Avadhani N G, Anandatheerthavarada H K (2006) Accumulation of amyloid precursor protein in the mitochondrial import channels of human Alzheimer's disease brain is associated with mitochondrial dysfunction. J Neurosci 26, 9057-9068.
[15] Manczak M, Anekonda T S, Henson E, Park B S, Quinn J, Reddy P H (2006) Mitochondria are a direct site of Abeta accumulation in Alzheimer's disease neurons: implications for free radical generation and oxidative damage in disease progression. Hum Mol Genet 15, 1437-1449.
[16] Reddy P H, Beal M F (2008) Amyloid beta, mitochondrial dysfunction and synaptic damage: implications for cognitive decline in aging and Alzheimer's disease. Trends Mol Med 14, 45-53.
[17] Lin M T, Simon D K, Ahn C H, Kim L M, Beal M F. (2002) High aggregate burden of somatic mtDNA point mutations in aging and Alzheimer's disease brain. Hum Mol Genet 11, 133-145.
[18] Coskun P E, Beal M F, Wallace D C (2004) Alzheimer's brains harbor somatic mtDNA control-region mutations that suppress mitochondrial transcription and replication. Proc Natl Acad Sci USA 101, 10726-1031.
[19] Chandrasekaran K, Giordano T, Brady D R, Stoll J, Martin L J, Rapoport S I (1994) Impairment in mitochondrial cytochrome oxidase gene expression in Alzheimer disease. Brain Res Mol Brain Res 24, 336-340.
[20] Reddy P H, McWeeney S, Park B S, Manczak M, Gutala R V, Partovi D, Jung Y, Yau V, Searles R, Mori M, Quin J F (2004) Gene expression profiles of transcripts in amyloid precursor protein transgenic mice: up-regulation of mitochondrial metabolism and apoptotic genes is an early cellular change in Alzheimer's disease. Hum Mol Genet 13, 1225-1240.
[21] Manczak M, Calkins M J, Reddy P H (2011) impaired mitochondrial dynamics and abnormal interaction of amyloid beta with mitochondrial protein Drp1 in neurons from patients with Alzheimer's disease: implications for neuronal damage. Hum Mol Genet 20, 2495-2509.
[22] Manczak M, Park B S, Jung Y, Reddy P H (2004) Differential expression of oxidative phosphorylation genes in patients with Alzheimer's disease: implications for early mitochondrial dysfunction and oxidative damage. Neuromolecular Med 5, 147-162.
[23] Manczak M, Mao P, Calkins M J, Cornea A, Reddy Aβ, Murphy M P, Szeto H H, Park B, Reddy P H (2010) Mitochondria-targeted antioxidants protect against amyloid-beta toxicity in Alzheimer's disease neurons. J Alzheimers Dis 20 Suppl 2, S609-S631.
[24] Calkins M J, Reddy P H (2011) Assessment of newly synthesized mitochondrial DNA using BrdU labeling in primary neurons from Alzheimer's disease mice: Implications for impaired mitochondrial biogenesis and synaptic damage. Biochim Biophys Acta 1812, 1182-1189.
[25] Calkins M J, Manczak M, Mao P, Shirendeb U, Reddy P H (2011) Impaired mitochondrial biogenesis, defective axonal transport of mitochondria, abnormal mitochondrial dynamics and synaptic degeneration in a mouse model of Alzheimer's disease. Hum Mol Genet 20, 4515-4529.
[26] Wang X, Su B, Siedlak S L, Moreira P I, Fujioka H, Wang Y, Casadesus G, Zhu X (2008) Amyloid-beta overproduction causes abnormal mitochondrial dynamics via differential modulation of mitochondrial fission/fusion proteins. Proc Natl Acad Sci USA 105, 19318-19323.
[27] Wang X, Su, B, Lee H G, Li X, Perry G, Smith M A, Zhu X (2009) Impaired balance of mitochondrial fission and fusion in Alzheimer's disease. J Neurosci 29, 9090-9103.
[29] Manczak M, Kandimalla R, Fry D, Sesaki H, Reddy P H (2016) Protective Effects of Reduced Dynamin-related Protein 1 Against Amyloid Beta-induced Mitochondrial Dysfunction and Synaptic Damage in Alzheimer's Disease. Hum Mol Genet in press
[30] Kandimalla, R., Manczak, M., Fry, D., Suneetha, Y., Sesaki, H. and Reddy, P. H. Suneetha Y, Sesaki H, Reddy P H (2016) Reduced Dynamin-related Protein 1 Protects Against Phosphorylated Tau-induced Mitochondrial Dysfunction and Synaptic Damage in Alzheimer's Disease. Hum. Mol. Genet in press
[31] Trushina E, Nemutlu E, Zhang S, Christensen T, Camp J, Mesa J, Siddiqui A, Tamura Y, Sesaki, H, Wengenack T M, Dzeja P P, Poduslo J F (2012) Defects in mitochondrial dynamics and metabolomic signatures of evolving energetic stress in mouse models of familial Alzheimer's disease. PLoS One, 7, e32737.
[32] Reddy P H (2014) Increased mitochondrial fission and neuronal dysfunction in Huntington's disease: implications for molecular inhibitors of excessive mitochondrial fission. Drug Discov Today 19, 951-955.
[33] Reddy P H (2014) Inhibitors of mitochondrial fission as a therapeutic strategy for diseases with oxidative stress and mitochondrial dysfunction. J Alzheimers Dis 40, 245-256
[34] Cassidy-Stone A, Chipuk J E, Ingerman E, Song C, Yoo C, Kuwana T, Kurth M J, Shaw J T, Hinshaw J E, Green D R, Nunnari J (2008) Chemical inhibition of the mitochondrial division dynamin reveals its role in Bax/Bak-dependent mitochondrial outer membrane permeabilization. Dev Cell. 14, 193-204.
[35] Macia E, Ehrlich M, Massol R, Boucrot E, Brunner C, Kirchhausen T (2008) Dynasore, a cell-permeable inhibitor of dynamin. Dev Cell. 2006 10, 839-850.
[36] Qi X, Qvit N, Su Y C, Mochly-Rosen D (2013) A novel Drp1 inhibitor diminishes aberrant mitochondrial fission and neurotoxicity. J Cell Sci126, 789-802.
[37] Qiu X, Cao L, Yang X, Zhao X, Liu X, Han Y, Xue Y, Jiang H, Chi Z (2013) Role of mitochondrial fission in neuronal injury in pilocarpine-induced epileptic rats. Neuroscience 245, 157-165.
[38] Ong S B, Subrayan S, Lim S Y, Yellon D M, Davidson S M, Hausenloy D J (2010) Inhibiting mitochondrial fission protects the heart against ischemia/reperfusion injury. Circulation. 121, 2012-2022.
[39] Zhang X, Yan H, Yuan Y, Gao J, Shen Z, Cheng Y, Shen Y, Wang R R, Wang X, Hu W W, Wang G, Chen Z (2013) Cerebral ischemia-reperfusion-induced autophagy protects against neuronal injury by mitochondrial clearance. Autophagy 9, 1321-33.
[40] Park S W, Kim K Y, Lindsey J D, Dai Y, Heo H, Nguyen D H, Ellisman M H, Weinreb R N, Ju W K (2011) A selective inhibitor of drpl, mdivi-1, increases retinal ganglion cell survival in acute ischemic mouse retina. Invest Ophthalmol Vis Sci. 52, 2837-2843.
[41] Xie N, Wang C, Lian Y, Zhang H, Wu C, Zhang Q (2013) A selective inhibitor of Drp1, mdivi-1, protects against cell death of hippocampal neurons in pilocarpine-induced seizures in rats. Neurosci Lett. 545, 64-68.
[42] Wappler E A, Institoris A, Dutta S, Katakam P V, Busija D W (2013) Mitochondrial dynamics associated with oxygen-glucose deprivation in rat primary neuronal cultures. PLoS One 8, e63206.
[43] Chlystun M, Campanella M, Law A L, Duchen M R, Fatimathas L, Levine T P, Gerke V, Moss S E (2013) Regulation of mitochondrial morphogenesis by annexin A6. PLoS One 8, e53774.
[44] Manczak M, Reddy P H (2015) Mitochondrial division inhibitor 1 protects against mutant huntingtin-induced abnormal mitochondrial dynamics and neuronal damage in Huntington's disease. Hum Mol Genet 24, 7308-7325.
[45] Silva D F, Selfridge J E, Lu J, E L, Roy N, Hutfles L, Burns J M, Michaelis E K, Yan S, Cardoso S M, Swerdlow R H (2013) Bioenergetic flux, mitochondrial mass and mitochondrial morphology dynamics in A D and MCI cybrid cell lines. Hum Mol Genet 22, 3931-3946.
[46] Reddy P H (2006) Amyloid precursor protein-mediated free radicals and oxidative damage: implications for the development and progression of Alzheimer's disease. J Neurochem 96, 1-13.
[47] Aleardi A M, Benard G, Augereau O, Malgat M, Talbot J C, Mazat J P, Letellier T, Dachary-Prigent J, Solaini G C, Rossignol R (2005) Gradual alteration of mitochondrial structure and function by beta-amyloids: importance of membrane viscosity changes, energy deprivation, reactive oxygen species production, and cytochrome c release. J Bioenerg Biomembr. 37, 207-225.
[48] Casley C S, Canevari L, Land J M, Clark J B, Sharpe M A (2002) Beta-amyloid inhibits integrated mitochondrial respiration and key enzyme activities. J Neurochem 80, 91-100.
[49] Casley C S, Land J M, Sharpe M A, Clark J B, Duchen M R, Canevari L (2005) Beta-amyloid fragment 25-35 causes mitochondrial dysfunction in primary cortical neurons. Neurobiol Dis 10, 258-267.
[50] Shirendeb U, Reddy Aβ, Manczak M, Calkins M J, Mao P, Tagle D A, Reddy P (2011) Abnormal mitochondrial dynamics, mitochondrial loss and mutant huntingtin oligomers in Huntington's disease: implications for selective neuronal damage. Hum Mol Genet 20, 1438-55.
[51] Shirendeb U P, Calkins M J, Manczak M, Anekonda V, Dufour B, McBride J L, Mao P, Reddy P H (2012) Mutant huntingtin's interaction with mitochondrial protein Drp1 impairs mitochondrial biogenesis and causes defective axonal transport and synaptic degeneration in Huntington's disease. Hum Mol Genet 21, 406-420.
[52] Song W, Chen J, Petrilli A, Liot G, Klinglmayr E, Zhou Y, Poquiz P, Tjong J, Pouladi M A, Hayden M R, Masliah E, Ellisman M, Rouiller I, Schwarzenbacher R, Bossy B, Perkins G, Bossy-Wetzel E (2011) Mutant huntingtin binds the mitochondrial fission GTPase dynamin-related protein-1 and increases its enzymatic activity. Nat Med 17, 377-382.
[53] Yin X, Manczak M, Reddy P H (2016) Mitochondria-targeted molecules MitoQ and SS31 reduce mutant huntingtin-induced mitochondrial toxicity and synaptic damage in Huntington's disease. Hum Mol Genet 25, 1739-1753.
[54] Wang H, Song P, Du L, Tian W, Yue W, Liu M, Li D, Wang B, Zhu Y, Cao C, Zhou J, Chen Q. Parkin ubiquitinates Drp1 for proteasome-dependent degradation: implication of dysregulated mitochondrial dynamics in Parkinson disease. J Biol Chem 286, 11649-11658.
[55] Wang X, Yan M H, Fujioka H, Liu J, Wilson-Delfosse A, Chen S G, Perry G, Casadesus G, Zhu X (2012) LRRK2 regulates mitochondrial dynamics and function through direct interaction with DLP 1. Hum Mol Genet 21, 1931-1944.
[56] Stafa K, Tsika E, Moser R, Musso A, Glauser L, Jones A, Biskup S, Xiong Y, Bandopadhyay R, Dawson V L, Dawson T M, Moore D J (2014) Functional interaction of Parkinson's disease-associated LRRK2 with members of the dynamin GTPase superfamily. Hum Mol Genet 23, 2055-2077.
[57] Sadeghian M, Mastrolia V, Rezaei Haddad A, Mosley A, Mullali G, Schiza D, Sajic M, Hargreaves I, Heales S, Duchen M R, Smith K J (2016) Mitochondrial dysfunction is an important cause of neurological deficits in an inflammatory model of multiple sclerosis. Sci Rep. 6:33249.
[58] Magrane J, Sahawneh M A, Przedborski S, Estevez A G, Manfredi G (2012) Mitochondrial dynamics and bioenergetic dysfunction is associated with synaptic alterations in mutant SOD1 motor neurons. J Neurosci 32, 229-242.

What is claimed is:

1. A method for preventing or treating a disease or condition with excessive fragmentation of mitochondria or mitochondrial dysfunction comprising, consisting essentially of, or consisting of:
identifying a subject suspected of needing treatment for excessive fragmentation of mitochondria or mitochondrial dysfunction; and
administering to the subject with an amount of a mitochondrial division inhibitor 1 sufficient to prevent or treat the excessive fragmentation of mitochondria or mitochondrial dysfunction.

2. The method of claim 1, wherein the mitochondrial division inhibitor 1 comprises a phenolic OH group that enhances antioxidant/anti-inflammatory and water solubility.

3. The method of claim 1, wherein the mitochondrial division inhibitor 1 is modified to be at least partially soluble in water.

4. The method of claim 1, wherein the disease or condition with excessive fragmentation of mitochondria or mitochondrial dysfunction is Alzheimer's, Parkinson's, multiple sclerosis, amyotrophic lateral sclerosis, or Huntington's Disease.

5. The method of claim 1, further comprising one or more pharmaceutically acceptable excipients, fillers, salts, or buffers.

6. The method of claim 1, wherein the disease is not associated with epilepsy and seizures, ischemia/reperfusion injury, oxygen glucose deprivation, or conditions associated with endosome aggregation and vesicle fusion during exocytosis.

7. The method of claim 1, wherein the mitochondrial division inhibitor 1 or active derivative thereof is administered to the subject at a dose of 1 to 120 mg/day/person.

8. The method of claim 1, wherein the mitochondrial division inhibitor 1 or active derivative thereof is administered to the subject at a dose of 10-60 mg daily.

9. The method of claim 1, wherein the mitochondrial division inhibitor 1 or active derivative thereof is administered to the subject via oral or parenteral administration.

10. The method of claim 1, wherein the mitochondrial division inhibitor 1 is 3-(2,4-Dichloro-5-methoxyphenyl)-2,3-dihydro-2-thioxo-4(1H)-quinazolinone, 3-(2,4-Dichloro-5-methoxyphenyl)-2-sulfanyl-4(3H)-quinazolinone, or 3-(2,4-dichloro-5-hydroxyphenyl)-2-thioxo-2,3-dihydroquinazolin-4(1H)-one.

11. The method of claim 1, wherein the mitochondrial division inhibitor 1 or active derivative thereof is a racemate, enantiomer, diastereomer, a mixture of enantiomer or a mixture of diastereomer.

12. The method of claim 1, wherein a pharmaceutically acceptable salt of mitochondrial division inhibitor 1 or active derivative thereof is formed from at least one of: an organic acid selected from formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phthalic acid, benzenesulphonic acid, toluenesulphonic acid, naphtalenesulphonic acid, or, methanesulphonic acid.

13. The method of claim 1, wherein the mitochondrial division inhibitor 1 or active derivative thereof is on the form of a tablets, capsules, pills, powders, granules, elixirs, tinctures, suspensions, syrups, emulsion, or formulated for intravenous administration.

14. A method of treating or preventing memory loss in a subject suffering from a memory loss-related disease or aging, comprising, consisting essentially of, or consisting of, administering an effective amount of an amount of a mitochondrial division inhibitor 1 or active derivative thereof sufficient to prevent or treat the excessive fragmentation of mitochondria or mitochondrial dysfunction.

15. The method of claim 14, wherein the mitochondrial division inhibitor 1 or active derivative thereof is administered to the subject at a dose of 1 to 120 mg/day/person.

16. The method of claim 14, wherein the mitochondrial division inhibitor 1 or active derivative thereof is administered to the subject at a dose of 10-60 mg daily.

17. The method of claim 14, wherein the mitochondrial division inhibitor 1 or active derivative thereof is administered to the subject via oral or parenteral administration.

18. The method of claim 14, wherein the mitochondrial division inhibitor 1 is 3-(2,4-Dichloro-5-methoxyphenyl)-2,3-dihydro-2-thioxo-4(1H)-quinazolinone, 3-(2,4-Dichloro-5-methoxyphenyl)-2-sulfanyl-4(3H)-quinazolinone, or 3-(2,4-dichloro-5-hydroxyphenyl)-2-thioxo-2,3-dihydroquinazolin-4(1H)-one.

19. The method of claim 14, wherein the mitochondrial division inhibitor 1 or active derivative thereof is a racemate, enantiomer, diastereomer, a mixture of enantiomer or a mixture of diastereomer.

20. The method of claim 14, wherein a pharmaceutically acceptable salt of mitochondrial division inhibitor 1 or active derivative thereof is formed from at least one of: an organic acid selected from formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phthalic acid, benzenesulphonic acid, toluenesulphonic acid, naphtalenesulphonic acid, or, methanesulphonic acid.

21. The method of claim 14, wherein the memory loss-related disease is dementia.

22. The method of claim 14, wherein the dementia is Alzheimer's disease.

23. The method of claim 14, wherein a reduction in memory loss is in a cognitively normal older adult.

24. The method of claim 14, wherein the mitochondrial division inhibitor 1 or active derivative thereof is on the form of a tablets, capsules, pills, powders, granules, elixirs, tinctures, suspensions, syrups, emulsion, or formulated for intravenous administration.

25. A method for protecting a subject from excessive fragmentation of mitochondria or mitochondrial dysfunction in neural cells comprising, consisting essentially of, or consisting of:
identifying a subject suspected of needing treatment for excessive fragmentation of mitochondria or mitochondrial dysfunction in neurons, wherein the subject is suspected of having or being at risk for Alzheimer's Disease; and
administering to the subject with an amount of a mitochondrial division inhibitor 1 sufficient to prevent the excessive fragmentation of mitochondria or mitochondrial dysfunction to prevent Alzheimer's Disease.

26. A partially water-soluble mitochondrial division inhibitor 1 having the formula 3-(2,4-dichloro-5-hydroxyphenyl)-2-thioxo-2,3-dihydroquinazolin-4(1H)-one.
